(19) United States
(12) Patent Application Publication
(10) Pub. No.: US 2025/0067653 A1
(43) Pub. Date: Feb. 27, 2025

(54) INFORMATION PROCESSING DEVICE AND INFORMATION PROCESSING SYSTEM

(71) Applicant: Sony Group Corporation, Tokyo (JP)

(72) Inventors: Akihide Higuchi, Kanagawa (JP); Koji Futamura, Kanagawa (JP); Fumitaka Otsuka, Kanagawa (JP)

(73) Assignee: Sony Group Corporation, Tokyo (JP)

(21) Appl. No.: 18/726,228

(22) PCT Filed: Jan. 6, 2023
(86) PCT No.: PCT/JP2023/000111

§ 371 Date: Jul. 2, 2024

(30) Foreign Application Priority Data
Jan. 17, 2022 (JP) 2022-005126

(51) Int. Cl. G01N 15/1429; G01N 15/10; G16B 40/10

(52) U.S. Cl. CPC G01N 15/1429; G16B 40/10; G01N 2015/1006

(57) ABSTRACT

An object of the present disclosure is to provide a technique for automatically proposing a better combination of fluorochrome-labeled antibodies. The present disclosure provides an information processing device including a processing unit configured to present a fluorophore candidate that is capable of being assigned to a biomolecule on the basis of fluorophore use history information, in-document fluorophore use result information, or both of them. The processing unit is configured to cause an output unit to display a selection window that prompts selection of a biomolecule to be analyzed in biological sample analysis, and the selection window is configured to select an analysis target biomolecule from among the biomolecules included in the fluorophore use history information, the in-document fluorophore use result information, or both of the fluorophore use history information and the in-document fluorophore use result information.

### TECHNICAL FIELD

The present disclosure relates to an information processing device and an information processing system. More specifically, the present disclosure relates to an information processing device and an information processing system, in which a method of assigning a fluorophore to a biomolecule is proposed.

### BACKGROUND ART

For example, a particle population such as cells, microorganisms, and liposomes is labeled with a fluorochrome, and the intensity and/or pattern of fluorescence generated from the fluorochrome excited by irradiating each particle of the particle population with laser light is measured, thereby measuring the characteristics of the particles. As a representative example of a particle analyzer that performs the measurement, a flow cytometer can be mentioned.

The flow cytometer is a device that irradiates particles flowing in a line in a flow path with laser light (excitation light) having a specific wavelength and detects fluorescence and/or scattered light emitted from each particle to analyze a plurality of particles one by one. The flow cytometer can convert light detected by a photodetector into an electrical signal, quantify the electrical signal, and perform statistical analysis to determine characteristics, for example, the type, size, structure, and the like of each particle.

Several techniques have been proposed so far regarding a method for selecting a fluorochrome used for labeling a particle population to be analyzed by the flow cytometer. For example, Patent Document 1 below discloses a reagent selection support apparatus for supporting selection of a reagent used for cell measurement. The reagent selection support apparatus includes an acquisition unit that acquires order information including a plurality of measurement items, a processing unit that determines a combination of a first fluorescent reagent used to measure a first target molecule corresponding to the plurality of measurement items and a second fluorescent reagent used to measure a second target molecule corresponding to a second measurement item different from the first measurement item on the basis of information reflecting the characteristics of the first target molecule and the characteristics of a first fluorochrome included in the first fluorochrome and information reflecting the characteristics of the second target molecule and the characteristics of a second fluorochrome included in the second fluorochrome, and an output unit that outputs the determined combination of the first fluorescent reagent and the second fluorescent reagent.

### CITATION LIST

### Patent Document

Patent Document 1: Japanese Patent Application Laid-Open No. 2019-203842

### SUMMARY OF THE INVENTION

### Problems to be Solved by the Invention

In order to label the particle population to be analyzed, a plurality of fluorochrome-labeled antibodies is often used. The determination process of the combination of the fluorochrome-labeled antibodies used in the analysis is also called panel design. The number of fluorochrome-labeled antibodies used in the analysis tends to increase, and as the number of fluorochrome-labeled antibodies increases, the panel design becomes more difficult. For example, about 20 or more panel designs may be built. For such a panel design, in a case where an optimal combination is proposed on the basis of information regarding all the commercially available fluorescent reagents, the calculation amount becomes very large. Furthermore, it is also required to build panel design that ensures the degree of freedom/flexibility for each user.

An object of the present disclosure is to provide a method for proposing a better combination of fluorochrome-labeled antibodies.

### Solutions to Problems

The present disclosure provides an information processing device including a processing unit configured to present a fluorophore candidate that is capable of being assigned to a biomolecule on the basis of fluorophore use history information, in-document fluorophore use result information, or both of them.

The fluorophore use history information may include a combination list of biomolecules and fluorophores, the combination list being used in biological sample analysis.

The in-document fluorophore use result information may include a combination list of biomolecules and fluorophores, the combination list being described in a document.

In an embodiment, the processing unit may be configured to cause an output unit to display a selection window that prompts selection of a biomolecule to be analyzed in biological sample analysis, and
the selection window may be configured to select an analysis target biomolecule from among the biomolecules included in the fluorophore use history information, the in-document fluorophore use result information, or both of them.

The processing unit may cause the output unit to display a fluorophore candidate that is capable of being assigned to the biomolecule selected in the selection window on the basis of the fluorophore use history information, the in-document fluorophore use result information, or both of them.

The processing unit may be configured to generate a combination list of fluorophores for biomolecules, and
the combination list may include a combination of the selected biomolecule and the fluorophore selected from among the fluorophore candidates.

The combination list may include a combination of the biomolecule and the fluorophore, which is specified by the processing unit, in addition to the combination of the selected biomolecule and the fluorophore selected from among the fluorophore candidates.

The processing unit may be configured to perform search for a biomolecule to be analyzed in biological sample analysis, and
the processing unit may perform search for the fluorophore use history information, the in-document fluorophore use result information, or both of them on the basis of an input keyword.

The fluorophore use history information, the in-document fluorophore use result information, or both of them may be associated with user information.

The information processing device may be configured to automatically collect a combination list of biomolecules and fluorophores, which is used in analysis processing of a biological sample analyzer, as the fluorophore use history information.

The information processing device may be configured to display a window that receives an input of the fluorophore use history information, a window that receives input of the in-document fluorophore use result information, or a window that receives input of both of them.

The information processing device may include a storage unit configured to store the fluorophore use history information, the in-document fluorophore use result information or both of them.

The processing unit may be configured to generate a combination list of fluorophores for biomolecules, and the processing unit may be configured to display an input box regarding whether to include a tandem dye in the combination list.

The information processing device may include a storage unit configured to store stock information of a fluorophore, price information, or both of the stock information and the price information.

Furthermore, the present disclosure provides an information processing system including a processing unit configured to present a fluorophore candidate that is capable of being assigned to a biomolecule on the basis of fluorophore use history information, in-document fluorophore use result information, or both of them.

### BRIEF DESCRIPTION OF DRAWINGS

FIG. 1 is a schematic diagram of a configuration example of a flow cytometer.

FIG. 2 is a diagram illustrating an experimental flow example in a case where the present disclosure is applied to flow cytometry.

FIG. 3 is a diagram illustrating a configuration example of an information processing device according to the present disclosure.

FIG. 4 is a diagram illustrating an example of a data structure of fluorophore use history information.

FIG. 5 is a diagram illustrating an example of a window for receiving an input of fluorophore use history information.

FIG. 6 is a diagram illustrating an example of a data structure of in-document fluorophore use result information.

FIG. 7 is a diagram illustrating an example of a window for receiving an input of in-document fluorophore use result information.

FIG. 8A is a diagram illustrating an example of a window for receiving an input of analysis target biomolecules.

FIG. 8B is a diagram for explaining elements included in the window.

FIG. 8C is a diagram for explaining contents displayed in the window.

FIG. 8D is a diagram for explaining contents displayed in the window.

FIG. 8E is a diagram for explaining contents displayed in the window.

FIG. 8F is a diagram for explaining contents displayed in the window.

FIG. 8G is a diagram for explaining contents displayed in the window.

FIG. 8H is a diagram for explaining contents displayed in the window.

FIG. 8I is a diagram for explaining contents displayed in the window.

FIG. 8J is a diagram for explaining contents displayed in the window.

FIG. 8K is a diagram for explaining contents displayed in the window.

FIG. 8L is a diagram for explaining contents displayed in the window.

FIG. 8M is a diagram for explaining contents displayed in the window.

FIG. 8N is a diagram for explaining contents displayed in the window.

FIG. 8O is a diagram for explaining an input box prompting selection for use of a tandem dye.

FIG. 8P is a diagram for explaining an input box prompting selection for use of stock information.

FIG. 8O is a diagram for explaining an input box prompting selection for use of price information.

FIG. 8R is a diagram illustrating an example of a button for executing a search.

FIG. 9A is a diagram illustrating an example of a window for receiving an input of analysis target biomolecules.

FIG. 9B is a diagram for explaining elements included in the window.

FIG. 9C is a diagram illustrating an example of an assignment result of a fluorophore.

FIG. 10 is a flowchart of processing executed by an information processing device according to the present disclosure.

FIG. 11A is a diagram for explaining information processing according to the present disclosure.

FIG. 11B is a diagram for explaining information processing according to the present disclosure.

FIG. 12 is a diagram illustrating a matrix of correlation coefficient square values.

FIG. 13 is a conceptual diagram for explaining how to assign fluorophores to biomolecules.

FIG. 14 is a diagram illustrating a fluorescence spectrum.

FIG. 15 is a diagram for explaining a stain index.

FIG. 16 is a diagram illustrating an example of a calculation result of an inter-fluorophore stain index.

FIG. 17 is a flowchart of processing executed by an information processing device according to the present disclosure.

FIG. 18 is a flowchart of separability evaluation processing.

FIG. 19 is a diagram illustrating an example of data of inter-fluorophore SI.

FIG. 20 is a diagram illustrating an example of a window in which a candidate fluorophore that substitutes a fluorophore having poor separation performance is displayed.

FIG. 21A is a diagram illustrating calculation results of inter-fluorophore SI.

FIG. 21B is a diagram illustrating calculation results of inter-fluorophore SI.

FIG. 22 is a flowchart of processing executed by an information processing device according to the present disclosure.

FIG. 23 is a diagram illustrating an example of an input reception window.

FIG. 24A is a diagram illustrating an example of a standardized inter-fluorophore SI list.

FIG. 24B is a diagram for explaining a method of calculating standardized inter-fluorophore SI.

FIG. 25A is a diagram illustrating an example of inter-fluorophore SSM.

FIG. 25B is a diagram for explaining a method of calculating inter-fluorophore SSM.

FIG. 26 is a diagram schematically illustrating an overall configuration of a biological sample analyzer.

FIG. 27 is a diagram illustrating a configuration example of an information processing system according to the present disclosure.

### MODE FOR CARRYING OUT THE INVENTION

Hereinafter, preferred modes for carrying out the present disclosure will be described. Note that embodiments to be described below illustrate representative embodiments of the present disclosure, and the scope of the present disclosure is not limited only to these embodiments. Note that the present disclosure will be described in the following order.

1. First Embodiment (Information Processing Device)

(1) Details of Problems of Invention

(2) Example of Flow of Experiment Performed Using Present Disclosure

(3) Description of First Embodiment

(3-1) Configuration Example of Information Processing Device

(3-2) Example of Processing by Processing Unit

(3-2-1) Acquisition of Fluorophore Use History Information

(3-2-2) Acquisition of In-Document Fluorophore Use Result Information

(3-2-3) Example of Generation Processing of Combination List of Antibodies and Fluorochromes

(3-2-3-1) Input Reception Processing of Biomolecule

(3-2-3-2) Candidate Presentation Processing Based on Fluorophore Use History Information

(3-2-3-3) Candidate Presentation Processing Based on In-Document Fluorophore Use Result Information

(3-2-3-4) Example of Selection Result

(3-2-4) Use of User Information

(3-2-5) Processing Related to Availability of Tandem Dye

(3-2-6) Display Order of Fluorophore

(3-2-7) Use of Stock Information or Price Information

(3-2-8) Search for Biomolecules

(3-3) Modification Example of Generation Processing of Combination List of Antibodies and Fluorochromes

(3-3-1) Example of Processing (Example of Processing in which Fluorophore Determined on basis of Use History Information or In-Document Result Information is Not Considered in Processing by Algorithm)

(3-3-2) Another Example of Processing (Example of Fluorescence Spectrum Used in Calculation of Correlation Coefficient)

(3-3-3) Still Another example of Processing (Example of Executing Separability Evaluation)

(3-3-4) Still Another Example of Processing (Processing Flow Considering Fluorophore Determined on Basis of Use History Information or In-Document Result Information in Processing by Algorithm)

(3-3-5) Still Another Example of Processing (Processing Flow in Case where Inter-Fluorophore Stain Index or Inter-Fluorophore Spillover Spreading Matrix is Used as Correlation Information)

(3-3-5-1) Case of Using Inter-Fluorophore Stain Index

(3-3-5-2) Case of Using Inter-Fluorophore Spillover Spreading Matrix

(3-4) Configuration Example of Biological Sample Analyzer

2. Second Embodiment (Information Processing System)

3. Third Embodiment (Information Processing Method)

4. Fourth embodiment (program)

### 1. First Embodiment (Information Processing Device)

### (1) Details of Problems of Invention

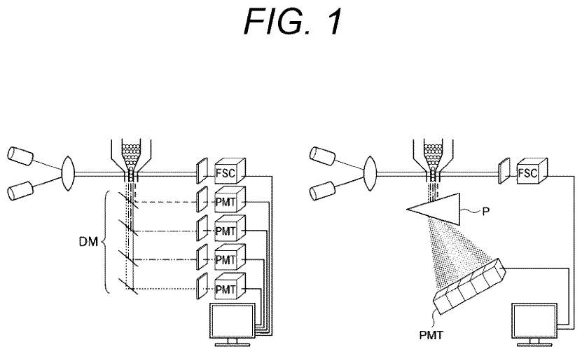

The flow cytometer can be roughly classified into a filter type and a spectral type, for example, from the viewpoint of an optical system for measuring fluorescence. The filter-type flow cytometer can adopt a configuration as illustrated in 1 of FIG. 1 in order to extract only target optical information from a target fluorochrome.

Specifically, light generated by irradiating the particles with light is branched into a plurality of pieces by a wavelength separation means DM such as a dichroic mirror, and passes through different filters, and then each branched light is measured by a plurality of detectors, for example, a photomultiplier tube PMT. That is, in the filter-type flow cytometer, multi-color fluorescence is detected by performing fluorescence detection for each wavelength band corresponding to each fluorochrome using a detector corresponding to each fluorochrome. At that time, in a case where a plurality of fluorochromes having close fluorescence wavelengths is used, fluorescence correction processing can be performed in order to calculate a more accurate fluorescence amount. However, in a case where a plurality of fluorochromes of which fluorescence spectra are very close to each other is used, leakage of fluorescence to a detector other than the detector to be detected increases, and thus an event in which fluorescence correction cannot be performed may also occur.

The spectral type flow cytometer analyzes the fluorescence amount of each particle by performing deconvolution (unmixing) on fluorescence data obtained by detecting light generated by irradiating the particles with light using spectrum information of the fluorochrome used for staining. As illustrated in 2 of FIG. 1, the spectral type flow cytometer disperses fluorescence by using a prism spectroscopic optical element P. Furthermore, in order to detect the dispersed fluorescence, the spectral type flow cytometer includes an array-type detector, for example, an array-type photomultiplier tube PMT instead of a large number of photodetectors of the filter-type flow cytometer. The spectral type flow cytometer is more likely to avoid the influence of leakage of fluorescence than the filter-type flow cytometer, and is more suitable for analysis using a plurality of fluorochromes.

In order to advance comprehensive interpretation in basic medicine and clinical fields, multicolor analysis using a plurality of fluorochromes has become widespread also in flow cytometry. However, when a large number of fluorochromes are used in one measurement as in multicolor analysis, as described above, fluorescence from a fluorochrome other than a target fluorochrome leaks into each detector, and analysis accuracy decreases in the filter-type flow cytometer. In a case where the number of colors is large, the problem of leakage of fluorescence can be solved to some extent by using the spectral type flow cytometer. However, in order to perform more appropriate multicolor analysis, an appropriate panel design (combination design of fluorochrome and antibody) is required.

In the related art, the panel design largely depends on user's experience and adjustment by trial and error. However, as the number of colors increases, particularly when the number of colors is about 20 or more, the panel design is often difficult.

Device manufacturers that sell flow cytometers, reagent manufacturers that sell antibodies with fluorochromes, and the like disclose web tools for panel design for promoting their products. However, as the number of colors increases, these web tools may not exhibit sufficient practicality.

### (2) Example of Flow of Experiment Performed Using Present Disclosure

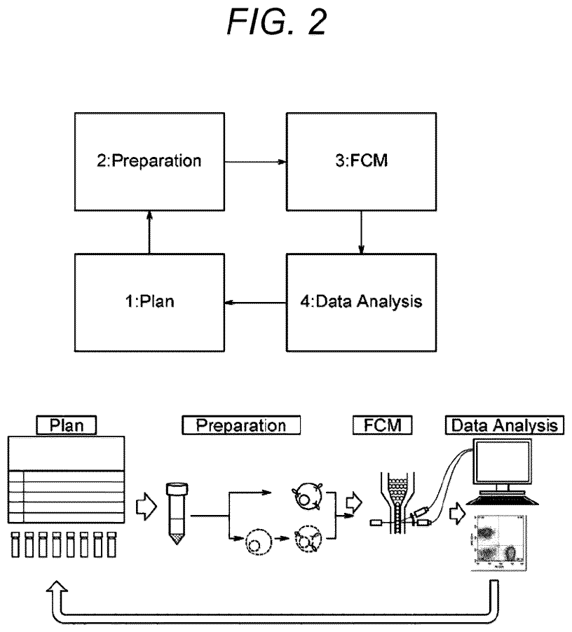

As described above, the present disclosure may be used to generate a list of combinations of antibodies and fluorophores, which is used in biological sample analysis such as flow cytometry. An experimental flow example in the case of applying the present disclosure in flow cytometry will be described with reference to FIG. 2.

The flow of the experiment using the flow cytometer is roughly classified into an experiment planning step (“1: Plan” in FIG. 2) of preparing an antibody reagent with a fluorescence index by examining a cell to be an experiment target and a method for detecting the cell, a sample preparation step (“2: Preparation” in FIG. 2) of actually staining and preparing the cell in a state suitable for measurement, an FCM measurement step (“3: FCM” in FIG. 2) of measuring the fluorescence amount of each stained cell by the flow cytometer, and a data analysis step (“4: Data Analysis” in FIG. 2) of performing various data processing so as to obtain a desired analysis result from data recorded by FCM measurement. Then, these steps can be repeated as necessary.

In the experiment planning step, first, which molecule (for example, an antigen, a cytokine, or the like) expression is used to determine a microparticle (mainly a cell) that is intended to be detected using a flow cytometer, that is, a marker used in detection of the microparticle is determined. Next, which fluorochrome is used to detect the marker is examined.

In the sample preparation step, the experiment target is first processed into a state suitable for FCM measurement. For example, cell separation and purification may be performed. For example, for immune cells derived from blood or the like, red blood cells are removed from the blood by hemolysis and density gradient centrifugation, and white blood cells are extracted. The extracted cell group of the target is stained using a fluorescently labeled antibody. At this time, in addition to the sample to be analyzed that is simultaneously stained with a plurality of fluorochromes, it is generally recommended to prepare a single stained sample that is stained with only one fluorochrome used as a reference at the time of analysis and a non-stained sample that is not stained at all.

In the FCM measurement step, when the microparticle is optically analyzed, first, excitation light is emitted from a light source of a light irradiation unit of the flow cytometer, and the microparticle flowing in the flow path is irradiated with the excitation light. Next, fluorescence emitted from the microparticle is detected by a detection unit of the flow cytometer. Specifically, by using a dichroic mirror, a bandpass filter, or the like, only light of a specific wavelength (target fluorescence) is separated from the light emitted from the microparticle, and the separated light is detected by a detector such as a 32 channel PMT. At this time, for example, fluorescence is dispersed using a prism, a diffraction grating, or the like, and light having different wavelengths are detected in each channel of the detector. Therefore, spectrum information of detection light (fluorescence) can be easily obtained. The microparticle to be analyzed is not particularly limited, and examples thereof include cells and microbeads.

The flow cytometer may have a function of recording fluorescence information of each fine particle acquired by FCM measurement together with scattered light information, time information, and position information other than the fluorescence information. The recording function can be mainly executed by a memory or a disk of a computer. In normal cell analysis, since analysis of several thousand to several million microparticles is performed under one experimental condition, it is necessary to record a large number of pieces of information in an organized state for each experimental condition.

In the data analysis step, light intensity data in each wavelength region detected in the FCM measurement step is quantified using a computer or the like, and the fluorescence amount (intensity) for each fluorochrome used is obtained. For this analysis, a correction method using a reference calculated from experimental data is used. The reference is calculated by statistical processing using two types of measurement data of microparticles stained with only one fluorochrome and measurement data of unstained microparticles. The calculated fluorescence amount can be recorded in a data recording unit provided in the computer together with information such as the name of the fluorescent molecule, the measurement date, and the type of microparticle. The fluorescence amount (fluorescence spectrum data) of the sample estimated by the data analysis is stored and displayed as a graph according to the purpose, and the fluorescence amount distribution of the microparticle is analyzed. For example, the proportion of cells to be detected included in the measured sample can be calculated by analyzing the fluorescence amount distribution.

The present disclosure may be applied to the panel design in the experiment planning step. In an embodiment, an information processing device according to the present disclosure presents a fluorophore candidate that can be assigned to a biomolecule on the basis of fluorophore use history information, in-document fluorophore use result information, or both of them. Thus, it is possible to prompt the user to select a fluorophore candidate for labeling the biomolecule, and it is possible to efficiently perform the panel design. Furthermore, for example, by presenting the fluorophore that has already been used, the user's desire is easily applied.

Furthermore, the information processing device according to the present disclosure may be configured to execute an algorithm for presenting a combination of the biomolecule and the fluorophore in addition to the presentation of such a fluorophore candidate. Thus, it is possible to cope with a case where the fluorophores cannot be assigned to all the biomolecules only by using the fluorophore use history information or the in-document fluorophore use result. Furthermore, it is also possible to search for a more appropriate fluorophore than the fluorophore presented in the fluorophore use history information or the in-document fluorophore use result.

Furthermore, as another example of particle analysis to which the present disclosure is applied, a microparticle sorting device that sorts microparticles in a closed space can be mentioned. For example, in order to determine whether to sort the microparticles, the device may include a chip that has a flow path through which the microparticles flow and in which the microparticles are sorted, a light irradiation unit that irradiates the microparticles flowing through the flow path with light, a detection unit that detects light generated by the light irradiation, and a determination unit that determines whether to sort the microparticles on the basis of information regarding the detected light. An example of the microparticle sorting device may include a device described in Japanese Patent Application Laid-Open No. 2020-041881.

Furthermore, the analysis to which the present disclosure is applied is not limited to particle analysis. That is, the present disclosure may be used in various types of processing in which assignment of a fluorophore to a biomolecule is required. For example, in microscopic analysis or observation of a cell sample or a tissue sample, such as multicolor fluorescence imaging, assignment processing of a fluorophore to a biomolecule according to the present disclosure may be performed in order to stain these samples. In recent years, the number of fluorophores used tends to increase also in fluorescence imaging, and the present disclosure can also be used in such analysis or observation.

### (3) Description of First Embodiment

An information processing device according to the present disclosure includes a processing unit that presents a fluorophore candidate that can be assigned to a biomolecule on the basis of fluorophore use history information, in-document fluorophore use result information, or both of them. By presenting the fluorophore candidate based on these pieces of information, the panel design can be efficiently performed, and for example, a range of fluorochrome candidate for searching for an optimum combination can be narrowed down. Thus, the amount of calculation for the panel design can be reduced, and the panel design can be performed in a shorter time. Furthermore, the presentation of the fluorophore candidate based on these pieces of information enables the panel design to which the user's desire is applied.

### (3-1) Configuration Example of Information Processing Device

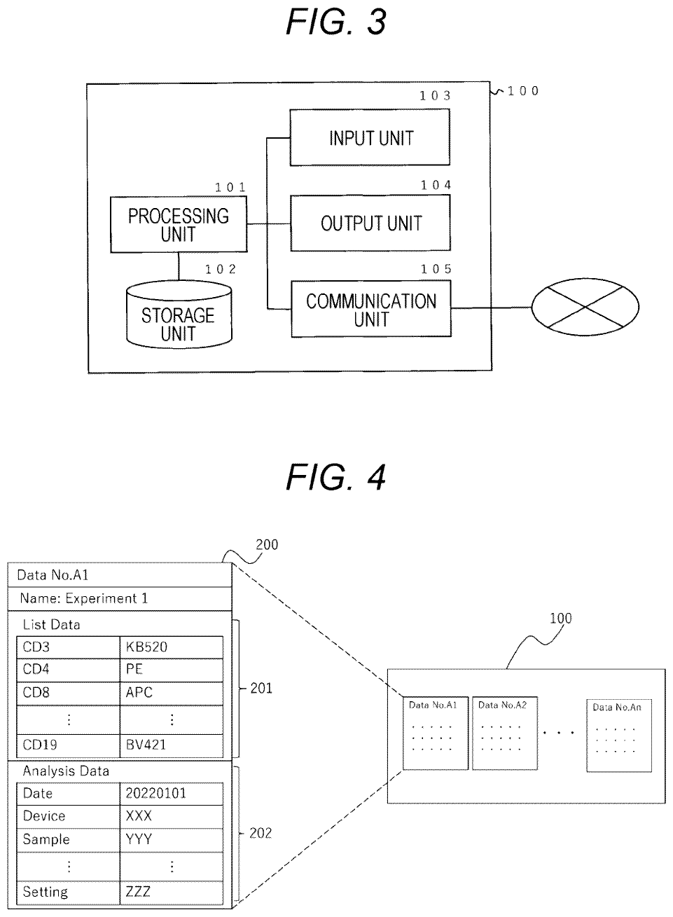

An example of the information processing device according to the present disclosure will be described with reference to FIG. 3. FIG. 3 is a block diagram of the information processing device. An information processing device 100 illustrated in FIG. 3 can include a processing unit 101, a storage unit 102, an input unit 103, an output unit 104, and a communication unit 105. The information processing device 100 may be configured by, for example, a general-purpose computer, may be a desktop computer, a laptop computer, or a tablet computer, or may be a server computer.

The processing unit 101 is configured to be able to present a fluorophore candidate that can be assigned to the biomolecule. The presentation may be performed on the basis of the fluorophore use history information, the in-document fluorophore use result information, or both of them. The specific processing of presentation will be described in detail below. The processing unit 101 can include, for example, a central processing unit (CPU) and a RAM. The CPU and the RAM may be connected to each other via, for example, a bus. An input/output interface may be further connected to the bus. The input unit 103, the output unit 104, and the communication unit 105 may be connected to the bus via the input/output interface.

The storage unit 102 stores various data. The storage unit 102 may be configured to be able to store, for example, data acquired in processing to be described later and/or data generated in processing to be described later, and for example, may include an information recording medium. Examples of these pieces of data include various kinds of data (for example, fluorophore use history information and in-document fluorophore use result information) received by the input unit 103, various kinds of data (for example, fluorophore use history information and in-document fluorophore use result information) received via the communication unit 105, and various kinds of data (for example, a combination list) generated by the processing unit 101, but are not limited to these kinds of data. Furthermore, the storage unit 102 can store an operating system (for example, Windows (registered trademark), UNIX (registered trademark), Linux (registered trademark), or the like), a program for causing the information processing device or the information processing system to execute the information processing method according to the present disclosure, and various other programs.

The input unit 103 may include an interface configured to be able to receive inputs of various kinds of data. For example, the input unit 103 may be configured to be able to receive various kinds of data input in processing to be described later. Examples of the data include biomolecule data and expression level data. The input unit 103 may include, for example, a mouse, a keyboard, and a touch panel, as a device that receives such an operation.

The output unit 104 may include an interface configured to be able to output various kinds of data. For example, the output unit 104 may be configured to be able to output various kinds of data generated in processing to be described later. Examples of the data include various kinds of data (for example, a combination list, and the like) generated by the processing unit 101, but the data is not limited to these pieces of data. The output unit 104 may include, for example, a display device as a device that · 22-outputs these pieces of data.

The communication unit 105 may be configured to connect the information processing device 100 to a network in a wired or wireless manner. With the communication unit 105, the information processing device 100 can acquire various kinds of data (for example, fluorophore use history information and in-document fluorophore use result information) via a network. The acquired data can be stored in, for example, the storage unit 102. The configuration of the communication unit 105 may be appropriately selected by those skilled in the art.

The information processing device 100 may include, for example, a drive (not illustrated) or the like. The drive can read data (for example, the various kinds of data described above) or a program (for example, the program described above) recorded in the recording medium and output the read data or program to the RAM. The recording medium is, for example, a microSD memory card, an SD memory card, or a flash memory, but is not limited to these.

### (3-2) Example of Processing by Processing Unit

Hereinafter, an example of the processing executed by the information processing device according to the present disclosure will be described.

### (3-2-1) Acquisition of Fluorophore Use History Information

In a case where analysis using a biomolecule (for example, a fluorochrome-labeled antibody) labeled with a fluorophore is performed, a biological sample analyzer (for example, a flow cytometer) that performs the analysis or a control device that controls the analyzer has information regarding a combination of one or more fluorophores used in the analysis and a molecule (for example, an antibody or the like) labeled with each fluorophore or a biomolecule (for example, an antigen or the like) captured by the molecule.

The information processing device according to the present disclosure may be configured to acquire information regarding the combination used in the analysis from the biological sample analyzer or the control device. The information processing device according to the present disclosure may use the acquired information as fluorophore use history information.

Furthermore, the information processing device may be configured to automatically collect a combination list of biomolecules and fluorophores used in analysis processing of the biological sample analyzer as the fluorophore use history information. For the automatic collection, for example, the biological sample analyzer may be configured to automatically transmit the combination list used in the analysis processing to the information processing device when the analysis processing is executed. Furthermore, for example, the information processing device can receive the transmitted combination list and store the combination list in the storage unit 102.

In the embodiment, in order to acquire the fluorophore use history information, the information processing device according to the present disclosure may be connected to the biological sample analyzer and the control device in a wired or wireless manner via a network or without a network. That is, the information processing device according to the present disclosure may receive the fluorophore use history information from the biological sample analyzer or the control device connected to the information processing device. In this embodiment, the information processing device according to the present disclosure can acquire the fluorophore use history information from, for example, a flow cytometer or a computer that controls the flow cytometer.

In another embodiment, the control device may be configured as the information processing device according to the present disclosure. In this case, the control device uses information regarding the fluorophore used in the analysis as fluorophore use history information when the analysis is performed by the biological sample analyzer. In this embodiment, for example, the information processing device according to the present disclosure may be configured as a computer that controls the flow cytometer, and can acquire the fluorophore use history information from the flow cytometer.

Moreover, in still another embodiment, the biological sample analyzer may be configured as the information processing device according to the present disclosure. That is, the biological sample analyzer may include the processing unit according to the present disclosure. In this embodiment, for example, the information processing device according to the present disclosure may be configured as the flow cytometer itself, and the fluorophore use history information can be stored in the flow cytometer.

The fluorophore use history information may include, for example, a combination list of biomolecules and fluorophores, which is used in the biological sample analysis.

The fluorophore use history information may further include analysis specification information for specifying the analysis. The analysis specification information may further include, for example, one or more pieces of data among data related to a date and time when the analysis is performed, data related to a device that performs the analysis, and data related to a sample subjected to the analysis.

An example of a data structure of the fluorophore use history information will be described with reference to FIG. 4. The information processing device 100 illustrated on the right side of FIG. 4 has fluorophore use history information (Data Nos. A1, A2, . . . , and An) related to the fluorophore used in each analysis. The fluorophore use history information may be stored in the information processing device, in particular, the storage unit 102 so as to be distinguishable for each analysis performed. Each fluorophore use history information includes a list of combinations of fluorophores and biomolecules (also referred to as a combination list of fluorophores for a biomolecule). For example, as illustrated in the left side of FIG. 4, fluorophore use history information 200 of Data No. A1 includes list data 201 indicting a list of combinations of biomolecules and fluorochromes assigned to the biomolecules. In this list, fluorochromes KB520, PE, and APC are assigned to biomolecules CD3, CD4, and CD8, respectively. Furthermore, as illustrated on the left side of FIG. 4, the fluorophore use history information may include analysis specification information (Analysis Data) 202. The analysis specification information includes data related to a date and time when the analysis is performed (Date: 20220101), data related to a device that performs the analysis (Device: XXX), and data related to a sample subjected to the analysis (Sample: YYY). Moreover, the analysis specification information may include data related to setting in the analysis (Settings: ZZZ).

Note that the data structure of the fluorophore use history information is not limited to the data structure illustrated in FIG. 4, and may be another data structure as long as the data structure includes a combination list of fluorophores for biomolecules. For example, since there is a case where there is a notation variability in the name of the fluorophore, the information regarding each fluorophore may be included in the fluorophore use history information as a code or a number instead of the name. In this case, the information processing device may include a data table for associating the code or number of the fluorophore with one or more names of the fluorophore. For example, in a case where the information processing device displays a fluorophore on a display device or the like on the basis of fluorophore use history information, the information processing device can display, with reference to the data table, a fluorophore name associated with the code or number, instead of the code or number. The same applies to the name of the biomolecule, and the information regarding each biomolecule may be included in the fluorophore use history information as a code or a number instead of the name.

The processing unit 101 of the information processing device 100 can be configured to be able to convert the fluorophore use history information into data having a predetermined data structure. An example of the data structure may be as described above.

The biological sample analyzer may have the fluorophore use history information with a different data structure depending on a type of device or a manufacturing company. As described above, by being converted into the predetermined data structure, the fluorophore use history information can be easily used in processing to be described later.

As described above, the information processing device 100 may be connected to the biological sample analyzer or the control device that controls the analyzer, and may receive the fluorophore use history information transmitted from these devices. That is, the information processing device 100 can receive the fluorophore use history information transmitted from other devices.

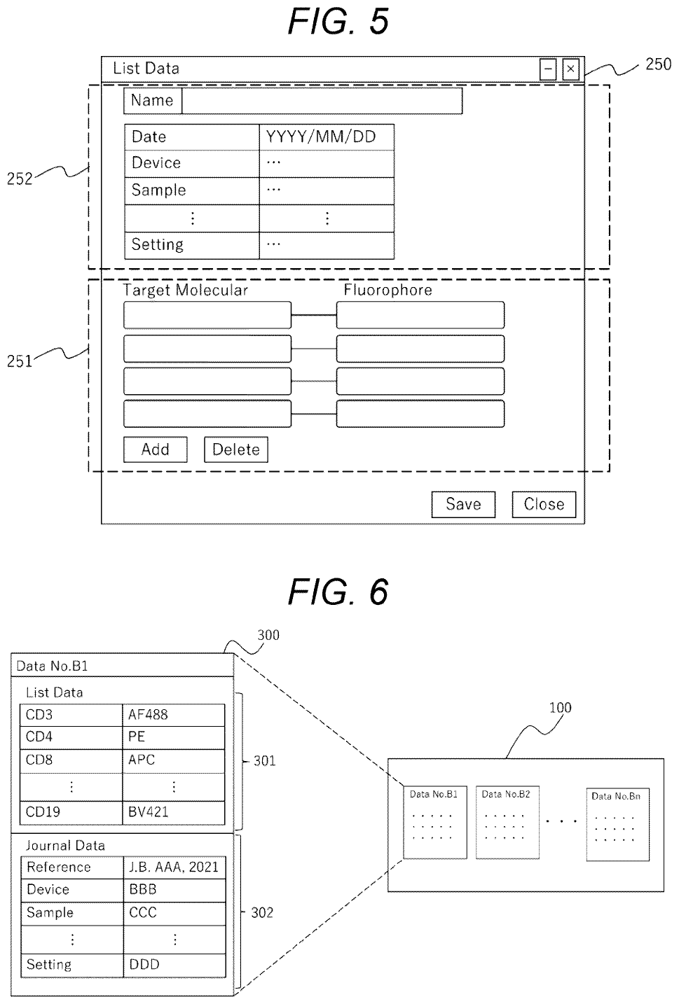

Furthermore, the fluorophore use history information may be directly input to the information processing device 100. That is, the information processing device 100 may be configured to be able to receive the input of the fluorophore use history information. In order to receive the input, the information processing device 100 may be configured to display a window that receives the input of the fluorophore use history information. An example of the window is illustrated in FIG. 5. A window 250 illustrated in FIG. 5 includes an area 251 for inputting a list of combinations of fluorophores and target biomolecules which are targeted by the antibodies labeled with the fluorophores. In FIG. 5, there are four combination input boxes in the area 251, that is, four pairs of fluorophore-biomolecule combinations are shown to be inputtable, but the number of inputtable combinations may be able to be increased or decreased by the user. For the increase or decrease, the window 250 may include a button (Add button) for adding a combination input box and a button (Delete button) for decreasing the number of the combination input boxes.

Moreover, the window 250 may include an area 252 for inputting data for specifying analysis using the combination list. The area 252 may include, for example, an analysis execution date input box (Date), an analyzer input box (Device), and a box (Sample) for inputting a sample subjected to the analysis. Furthermore, the area 252 may include a box for inputting other information, for example, a box (Settings) for inputting analysis setting.

Moreover, for example, the window 250 may include buttons for causing the information processing device 100 to execute processing on the input data or a window operation, such as a button (Save button) for storing the input data in the information processing device 100 (particularly, the storage unit 102) and a button (Close button) for closing the window.

The storage unit 102 of the information processing device 100 may be configured to be able to store the fluorophore use history information. Preferably, the storage unit can store the fluorophore use history information with the predetermined data structure described above.

### (3-2-2) Acquisition of In-Document Fluorophore Use Result Information

The contents and results of analysis using a biomolecule labeled with a fluorophore (for example, a fluorochrome-labeled antibody) are often described in the document such as papers (particularly, an academic document). That is, such a document describes information regarding a combination of one or more fluorophores and a molecule (for example, an antibody or the like) labeled with each fluorophore or a biomolecule (for example, an antigen or the like) captured by the molecule.

The information processing device according to the present disclosure may be configured to be able to receive an input of information regarding a combination of a biomolecule and a fluorophore described in such an academic document. The information processing device according to the present disclosure uses the information as in-document fluorophore use result information (also referred to as in-document fluorophore use result information).

The in-document fluorophore use result information may include, for example, a combination list of biomolecules and fluorophores, which is described in the document.

The in-document fluorophore use result information may further include document specification information for specifying the document. The document specification information may further include, for example, one or more pieces of data among data related to bibliographic items of the document, data related to a device that performs the analysis described in the document, and data related to a sample subjected to the analysis.

An example of a data structure of the in-document fluorophore use result information will be described with reference to FIG. 6. As illustrated on the right side of FIG. 6, the information processing device 100 has in-document fluorophore use result information (Data Nos. B1, B2, . . . , and Bn) related to the fluorophore described in each document. The in-document fluorophore use result information may be stored in the information processing device, in particular, the storage unit 102, so as to be distinguishable for each analysis described in the document. Each in-document fluorophore use result information includes a list related to combinations of fluorophores and biomolecules (also referred to as a combination list of fluorophores for biomolecules). For example, as illustrated in the left side of FIG. 6, in-document fluorophore use result information 300 of Data No. B1 includes list data 301 indicating a list of combinations of biomolecules and fluorochromes assigned to the biomolecules. In this list, fluorochromes AF488, PE, and APC are assigned to biomolecules CD3, CD4, and CD8, respectively. Furthermore, as illustrated on the left side of FIG. 6, the in-document fluorophore use result information may include document specification information (Journal Data) 302. The document specification information includes data related to bibliographic items for specifying the document (and analysis in the document) (Reference: J.B. AAA, 2021), data related to a device that performs the analysis described in the document (Device: BBB), and data related to a sample subjected to the analysis (Sample: CCC). Moreover, the analysis specification information may include data related to setting in the analysis (Setting: DDD).

Note that the data structure of the in-document fluorophore use result information is not limited to the data structure illustrated in FIG. 6, and may be another data structure as long as the data structure includes a combination list of fluorophores for biomolecules. As described above, the information regarding each biomolecule and each biomolecule may be included in the in-document fluorophore use result information as a code or a number instead of the name.

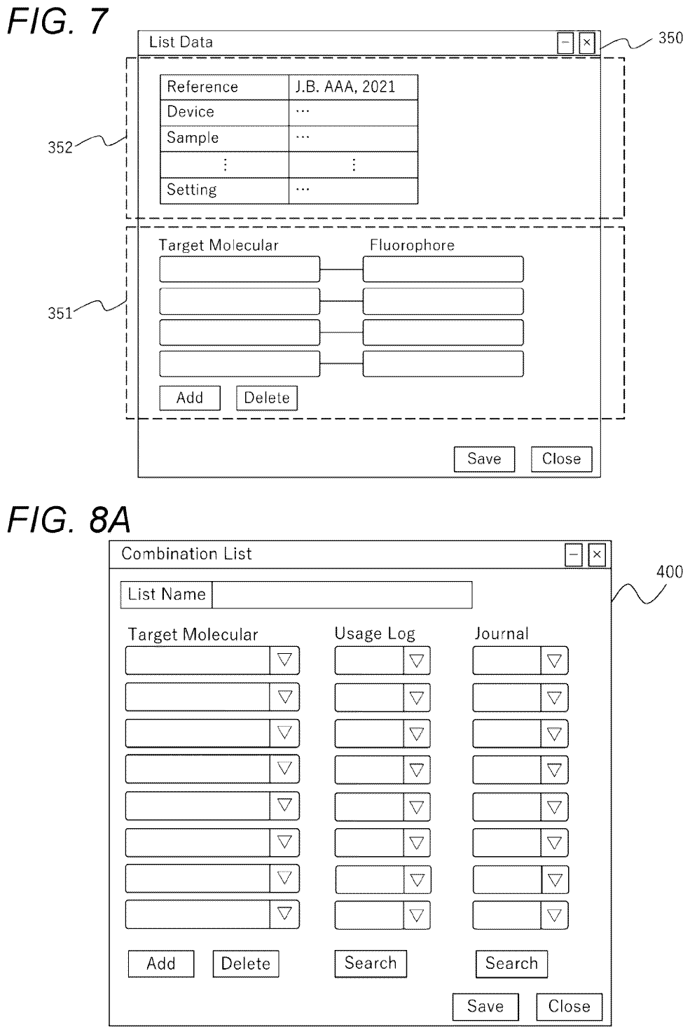

Furthermore, the information processing device 100 may be configured to be able to receive the input of the in-document fluorophore use result information. In order to receive the input, the information processing device 100 may be configured to display a window that receives the input of the fluorophore use history information. An example of the window is illustrated in FIG. 7. A window 350 illustrated in FIG. 7 includes an area 351 for inputting a list of combinations of fluorophores and target biomolecules which are targeted by the antigens labeled with the fluorophores. In FIG. 7, there are four combination input boxes in the area 351, that is, four pairs of fluorophore-biomolecule combinations are shown to be inputtable, but the number of inputtable combinations may be able to be increased or decreased by the user. For the increase or decrease, the window may include a button (Add button) for adding a combination input box and a button (Delete button) for decreasing the number of the combination input boxes.

Moreover, the window 350 may include an area 352 for inputting data for specifying a document in which the combination list is described. The area 352 may include, for example, an input box (Reference) for inputting bibliographic information of a document, an analyzer input box (Device), and a box (Sample) for inputting a sample subjected to the analysis. Furthermore, the area 352 may include a box for inputting other information, for example, a box (Setting) for inputting analysis setting. Moreover, for example, the window 350 may include buttons for causing the information processing device 100 to execute processing on the input data or a window operation, such as a button (Save button) for storing the input data in the information processing device 100 (particularly, the storage unit 102) and a button (Close button) for closing the window.

### (3-2-3) Example of Generation Processing of Combination List of Antibodies and Fluorochromes

The information processing device according to the present disclosure may be configured to generate a combination list of fluorophores for biomolecules. In the generation of the combination list, the fluorophore use history information described above, the in-document fluorophore use result information, or both of them can be used. Thus, it is possible to generate a combination list applying the desire of the user. Furthermore, the processing of generating the combination list can be executed more efficiently. That is, according to the present disclosure, a combination list of fluorophores for biomolecules can be generated more accurately and more efficiently. Hereinafter, the processing of generating a combination list of fluorophores for biomolecules, which uses the fluorophore use history information and/or the in-document fluorophore use result information, will be described.

### (3-2-3-1) Input Reception Processing of Biomolecule

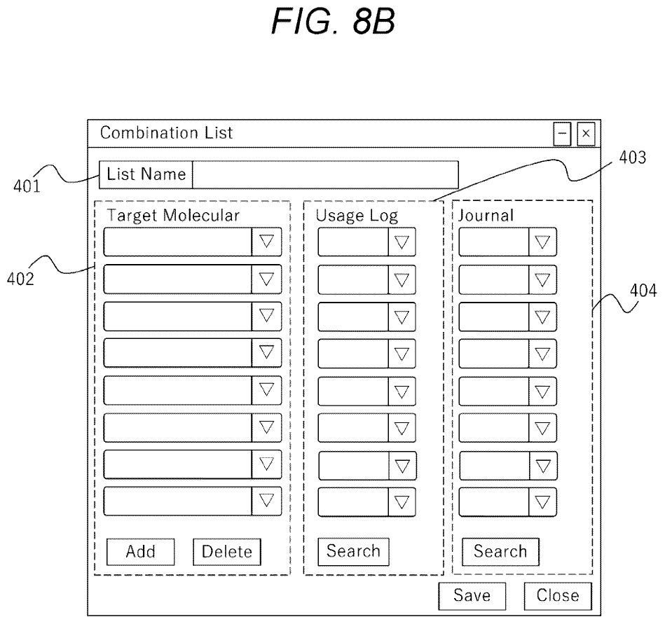

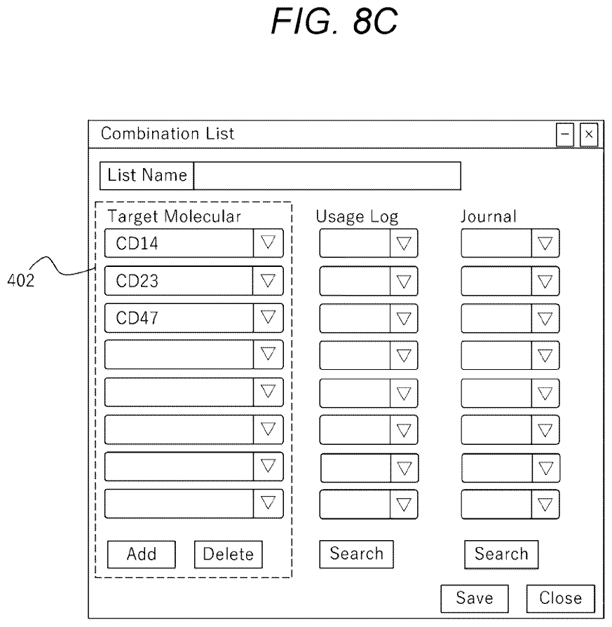

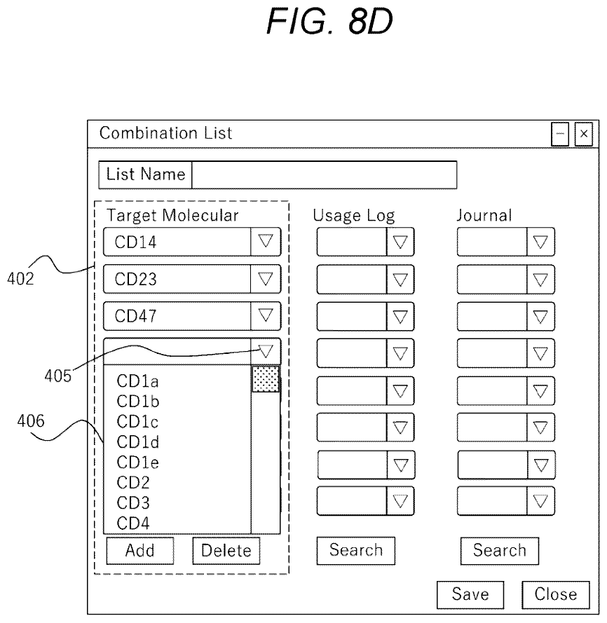

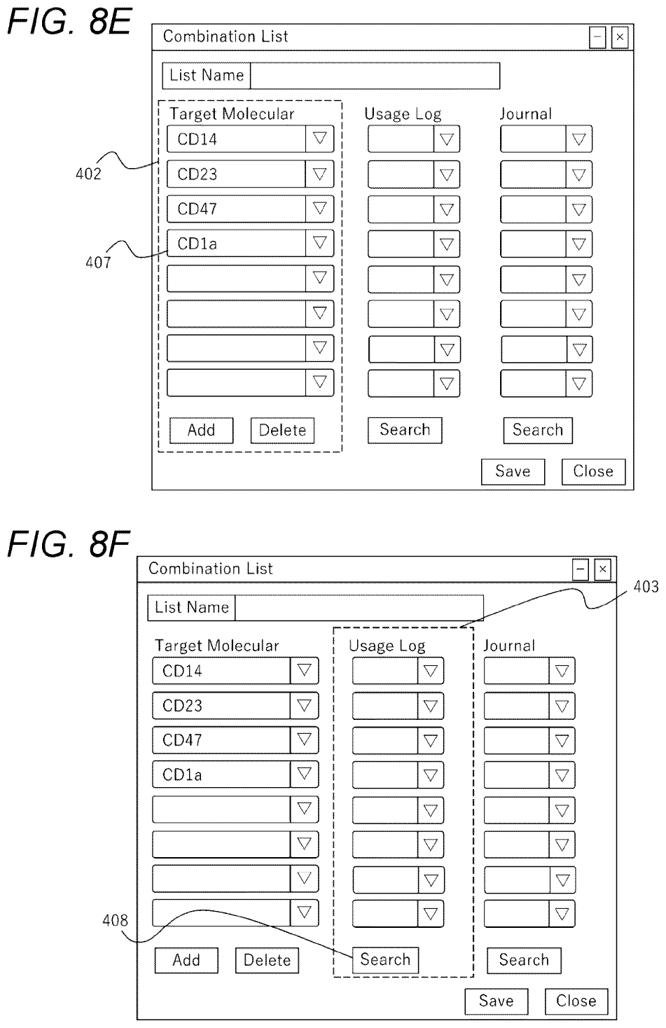

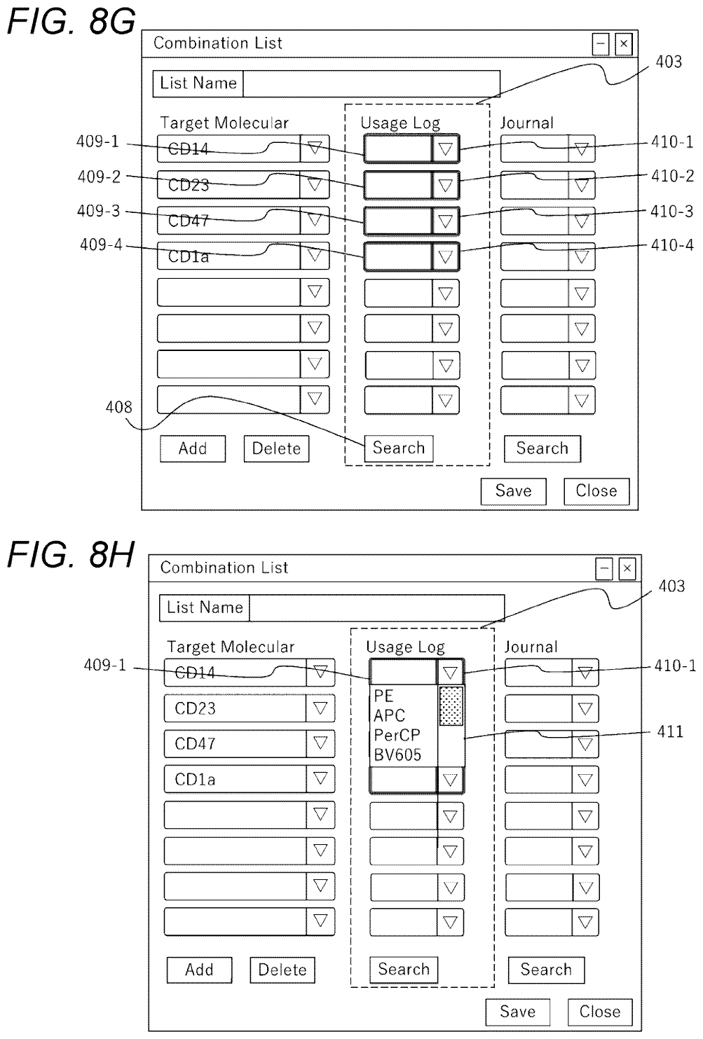

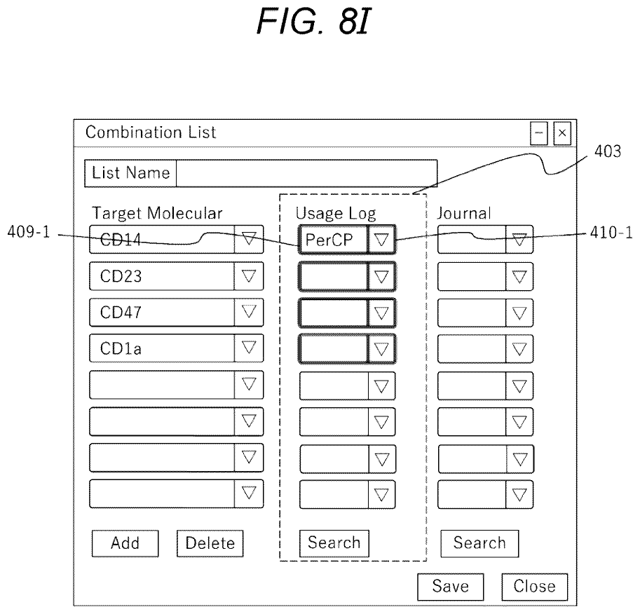

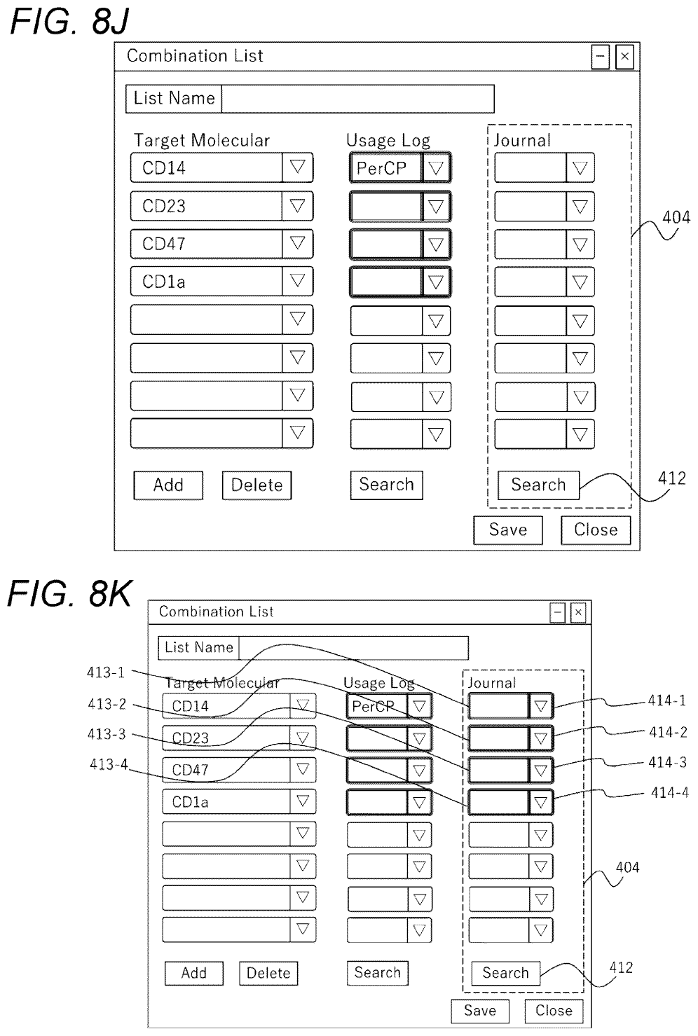

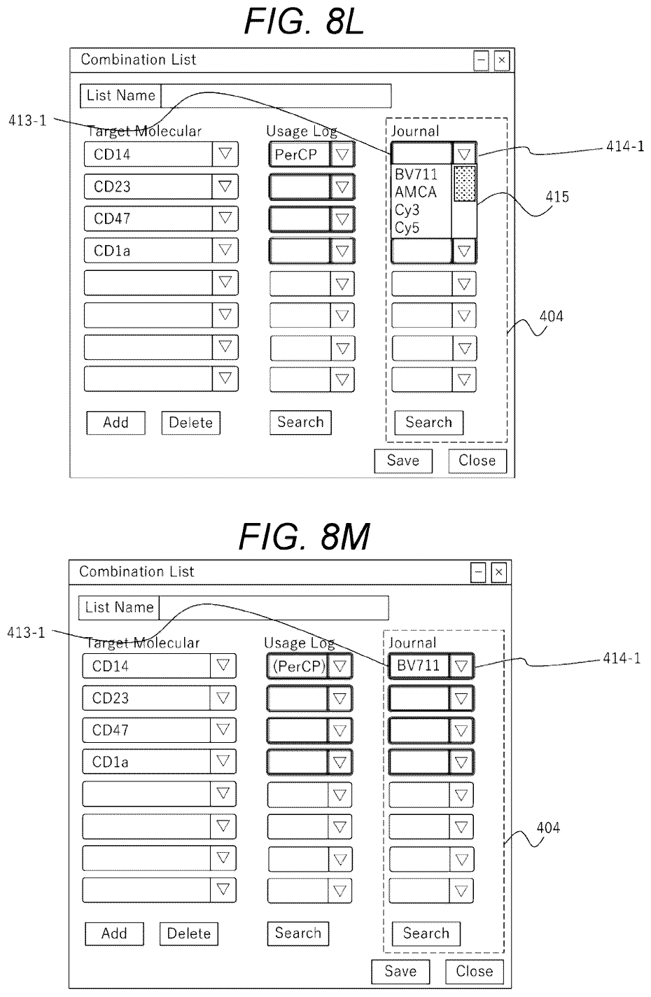

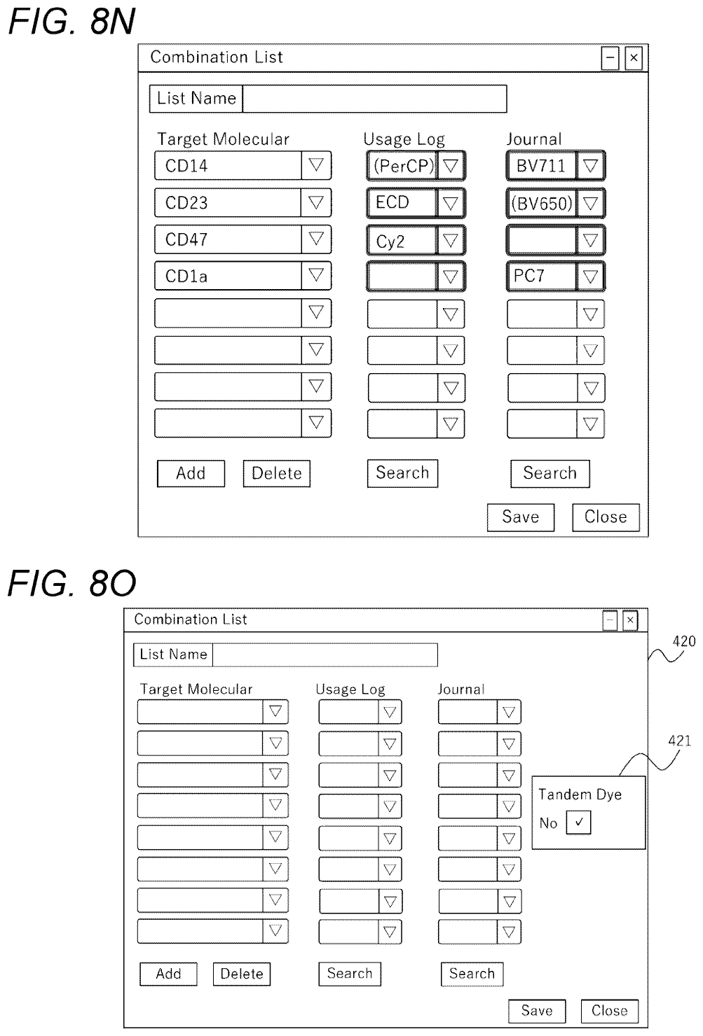

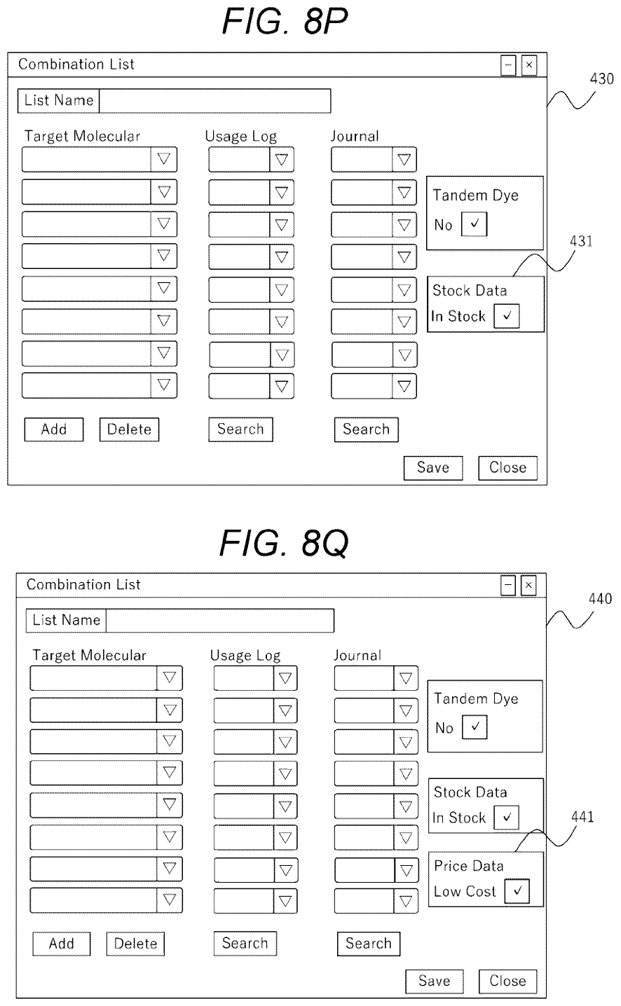

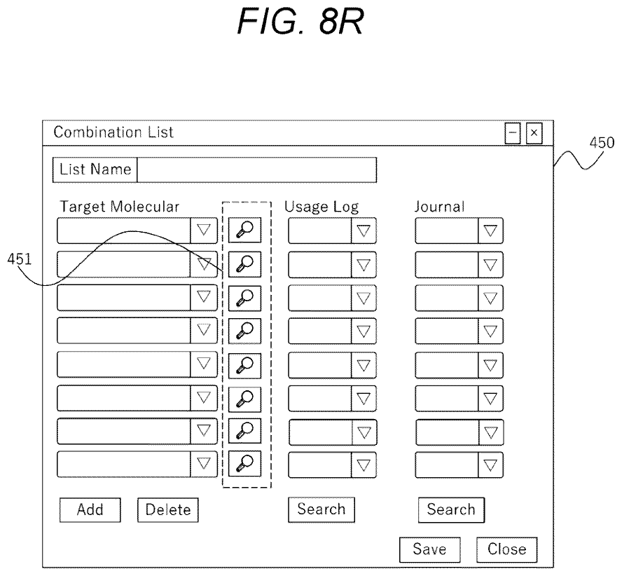

The information processing device according to the present disclosure may be configured to display a window that receives an input of a biomolecule (for example, an antigen) to be analyzed. An example of the window displayed by the information processing device according to the present disclosure will be described with reference to FIG. 8A. A window 400 illustrated in FIG. 8A is configured to receive an input of an analysis target biomolecule. As illustrated in FIG. 8B, the window includes a list name input area 401 to which the name of the combination list is input, a biomolecule input area 402 to which the analysis target biomolecule is input, a use information display area 403 in which the fluorophore use history information is displayed and the fluorophore is selected, and a document information display area 404 in which the in-document fluorophore use result information is displayed and the fluorophore is selected. As described above, the processing unit 101 may be configured to be able to display, on the output unit, a selection window that prompts selection of a biomolecule to be analyzed in biological sample analysis.

In the list name input area 401, the name of the combination list to be generated is input by the user. The information processing device 100 can store the name input to the area as the name of the combination list.

In the biomolecule input area 402, the name of the biomolecule to be analyzed may be input, or in the biomolecule input area 402, the name of the biomolecule can be selected from a biomolecule candidate list. In FIG. 8A, eight name input boxes are described.

For example, as illustrated in FIG. 8C, the user may input characters and the name of the biomolecule in each of these name input boxes. In FIG. 8C, the names of surface markers CD14, CD23, and CD47 are input in three input boxes, respectively.

These name input boxes may be configured to be able to select an analysis target biomolecule. In FIG. 8C, a biomolecule list display button 405 is provided in each name input box. When the button is selected, the information processing device displays a biomolecule candidate list 406 as illustrated in FIG. 8D. As illustrated in FIG. 8D, the biomolecule candidate list 406 may be displayed in a list box displayed when the button is selected. As illustrated in FIG. 8E, when any one from the list is selected, the biomolecule is input to a name input box 407 in which the button is selected.

### (3-2-3-2) Candidate Presentation Processing Based on Fluorophore Use History Information

In the use information display area 403, the processing unit 101 presents a fluorophore candidate that can be assigned to each biomolecule input in the biomolecule input area 402 on the basis of the fluorophore use history information.

For example, after the input of the biomolecule is completed in the biomolecule input area 402, the user selects use history information acquisition button 408 (Search button) illustrated in FIG. 8F. When the button is selected, the processing unit 101 specifies the fluorophore combined with each biomolecule input to the biomolecule input area 402 in the fluorophore use history information described in (3-2-1) described above. Then, the processing unit 101 generates a list including the specified fluorophores. Thus, a list of the fluorophore that is combined with each biomolecule is generated. For example, a list of a fluorochrome that has been used to label each antibody capturing each antigen input to the biomolecule input area 402 is generated.

The fluorophore list generated in this manner is also referred to as a fluorophore use history list in the present description.

In order to indicate that the fluorophore use history list has been generated, as illustrated in FIG. 8G, the processing unit 101 may highlight fluorophore use history display boxes 409-1 to 409-4 in which the list can be displayed when the generation of the fluorophore use history list is completed. In FIG. 8G, the lines of the fluorophore use history display boxes 409-1 to 409-4 in which the list can be displayed are thicker than those of the other boxes, but a highlighting method is not limited thereto, and the method may be, for example, changing color of each box or blinking each box.

The processing unit 101 can update display data of the window such that the list box having the fluorophore use history list corresponding to each input biomolecule can be displayed in each fluorophore use history display box next to the input box of each biomolecule. The processing unit 101 displays a fluorophore use history list 411 when a fluorophore use history list display button 410-1 illustrated in FIG. 8H is selected. The list can be displayed in association with the fluorophore use history display box 409-1, for example, as illustrated in FIG. 8H. Similarly, for the other display buttons 410-2 to 410-4, the fluorophore use history list is displayed when each button is selected. Thus, it is possible to prompt the user to select the fluorophore. An example of a window after the fluorophore is selected by the user is illustrated in FIG. 8I. In FIG. 8I, PerCP is selected as the fluorophore to be combined with CD14.

In the embodiment, the information processing device according to the present disclosure may present a fluorophore candidate on the basis of autofluorescence information or device noise information. The autofluorescence information may be, for example, information regarding autofluorescence of a sample, and in particular, may be information regarding autofluorescence of particles (in particular, cells) contained in the sample. The processing unit 101 excludes, for example, a fluorophore that generates fluorescence similar to autofluorescence included in the autofluorescence information from the fluorophore use history list. Thus, the fluorophore that generates fluorescence similar to autofluorescence is excluded from the candidates. The device noise information may be used similarly to the autofluorescence information. The processing unit 101 excludes, for example, the fluorophore that generates fluorescence similar to the device noise information from the fluorophore use history list.

For example, the processing unit 101 can use the autofluorescence information and/or the device noise information in the processing of generating a combination list of antibodies and fluorochromes described in (3-3) below. More specifically, the processing unit 101 uses each of the autofluorescence information and/or the device noise information as a fluorophore in the generation processing. By using the autofluorescence information and/or the device noise information in this manner, a combination list considering these pieces of information is generated.

### (3-2-3-3) Candidate Presentation Processing Based on In-Document Fluorophore Use Result Information

In the document information display area 404, the processing unit 101 presents a fluorophore candidate that can be assigned to each biomolecule input in the biomolecule input area 402 on the basis of the in-document fluorophore use result information.

For example, after the input of the biomolecule is completed in the biomolecule input area 402, a user selects document information acquisition button 412 (Search button) illustrated in FIG. 8J. When the button is selected, the processing unit 101 specifies the fluorophore combined with each biomolecule input to the biomolecule input area 402 in the in-document fluorophore use result information described in (3-2-1) described above. Then, the processing unit 101 generates a list including the specified fluorophores. Thus, a list of the fluorophore that is combined with each biomolecule is generated. For example, a list of a fluorochrome described in the document that the fluorochrome has been used to label each antibody capturing each antigen input to the biomolecule input area 402 is generated.

The fluorophore list generated in this manner is also referred to as an in-document use result list in the present description.

In order to indicate that the in-document use result list has been generated, as illustrated in FIG. 8K, the processing unit 101 may highlight in-document use result display boxes 413-1 to 413-4 in which the list can be displayed when the generation of the in-document use result list is completed. In FIG. 8K, the lines of the in-document use result display boxes 413-1 to 413-4 in which the list can be displayed are thicker than those of the other boxes, but a highlighting method is not limited thereto, and the method may be, for example, changing color of each box or blinking each box.

The processing unit 101 can update display data of the window such that the list box having the in-document use result list corresponding to each input biomolecule can be displayed in each in-document use result display box next to the input box of each biomolecule. The processing unit 101 displays an in-document use result list 415 when a fluorophore use history list display button 414-1 illustrated in FIG. 8L is selected. The list can be displayed in association with the in-document use result display box 413-1, for example, as illustrated in FIG. 8L. Similarly, for the other display buttons 414-2 to 414-4, the in-document use result list is displayed when each button is selected. Thus, it is possible to prompt the user to select the fluorophore. An example of a window after the fluorophore is selected by the user is illustrated in FIG. 8M. In FIG. 8M, BV711 is selected as the fluorophore to be combined with CD14.

When the fluorophore is selected in the in-document use result display box 413-1, the selection result of the fluorophore in the fluorophore use history display box may be invalidated. Thus, it is possible to prevent two fluorophores from being selected for one biomolecule. In FIG. 8M, characters of PerCP are enclosed in parentheses to indicate that the selection result of PerCP described in (3-2-3-2) above is canceled, but the method for indicating cancellation is not limited thereto.

Note that for each biomolecule, the selected fluorophore may be displayed in both the fluorophore use history display box and the in-document use result display box. In this case, the processing unit 101 may perform display that prompts the user to select which fluorophore to use.

### (3-2-3-4) Example of Selection Result

For other biomolecules, the fluorophores are similarly selected. An example of the selection result is illustrated in FIG. 8N. As illustrated in FIG. 8N, BV711, ECD, CY2, and PC7 are selected respectively for CD14, CD23, CD47, and CDla.

In this manner, a list including a combination of the biomolecule CD14 and the fluorophore BV711, a combination of the biomolecule CD23 and the fluorophore ECD, a combination of the biomolecule CD47 and the fluorophore CY2, and a combination of CDla and the fluorophore PC7 is generated. In this manner, the processing unit 101 may be configured to generate a combination list of the fluorophores for the biomolecules. The combination list includes a combination of the selected biomolecule and the fluorophore selected from the fluorophore candidates. The processing unit 101 can store the combination list of the fluorophores for the biomolecules generated in this manner in the storage unit 102.

The information processing device according to the present disclosure may execute order processing for purchasing a set of biomolecule capturing substances (for example, fluorochrome-labeled antibodies) labeled with a fluorophore on the basis of the generated combination list or at least some of the biomolecule capturing substances included in the set.

The user prepares a set of fluorophore-labeled antibodies according to the list. The set may be used in analysis processing of the biological sample analyzer, for example, the flow cytometer.

### (3-2-4) Use of User Information

In the embodiment, the fluorophore use history information or the in-document fluorophore use result information or both of them may be associated with the user information. The user information may include, for example, user specification information such as a user ID.

The information processing device according to the present disclosure can be used in a state where the user logs in by using a predetermined ID or the like. In this case, the user information of the logged-in user and the fluorophore use history information, the in-document fluorophore use result information or both of them are associated with each other, and thus the optimization processing described in (3-2-3) above can be executed on the basis of only the associated information. Thus, the user's preference is accurately applied.

For example, the fluorophore use history information may be protocol information of processing executed by the biological sample analyzer, and the protocol information may include the combination list. Such protocol information may be data associated with the user information. Since such protocol information is independently built by each user, the user's desire is applied. Therefore, the user's preference is accurately applied by using such protocol information.

Furthermore, the user information may be used by a biological sample analyzer that transmits the fluorophore use history information to the information processing device according to the present disclosure or a control device of the analyzer. The biological sample analyzer or the analyzer also can be used in a state where the user logs in by using a predetermined ID or the like. Thus, the biological sample analyzer or the analyzer can hold the fluorophore use history information in a state of being associated with the user information, and can transmit the fluorophore use history information to the information processing device according to the present disclosure in a state of being associated with the user information. Thus, the information processing device according to the present disclosure can hold the transmitted fluorophore use history information in a state of being associated with the user information.

### (3-2-5) Processing Related to Availability of Tandem Dye

In the embodiment, the information processing device according to the present disclosure may be configured to prompt selection for use of a tandem dye. For example, the processing unit 101 may be configured to display an input box related to whether to include a tandem dye in the combination list. The input box may be configured to include, for example, any one or more of selection items of using a tandem dye, not using a tandem dye, preferentially using a fluorophore other than the tandem dye over the tandem dye, and preferentially using the tandem dye over the fluorophore other than the tandem dye. Thus, it is possible to manage the user's preference for the use of the tandem dye.

For example, the processing unit 101 can display a window 420 as illustrated in FIG. 8O. The window is the same as the window 400 described above except that the window has an input box 421 that prompts selection for use of the tandem dye. In FIG. 8O, the input box includes an input box (No box) for selecting not to use the tandem dye and an input box (Acceptable box) for selecting preferential use of a fluorophore other than the tandem dye over the tandem dye.

In FIG. 8O, the No box is checked. In a case where the No box is checked, the processing unit 101 excludes, for example, the tandem dye from the fluorophore use history list and/or the in-document use result list. Thus, the tandem dye is excluded from the candidates. In a case where the No box is not checked, the processing unit 101 includes, for example, the tandem dye in the fluorophore use history list and/or the in-document use result list.

### (3-2-6) Display Order of Fluorophore

In the embodiment, the information processing device according to the present disclosure may be configured to be able to change a description order of a fluorophore group in the fluorophore use history list and/or the in-document use result list. For example, the processing unit 101 displays a box for causing the user to select to change the description order to any of the order of price, the order of use frequency, the order of wavelength (the order of excitation wavelength or the order of fluorescence wavelength), or the alphabetical order. The box may exist, for example, in the window 400. Thus, it is easy to select the fluorophore.

For example, in order to display the fluorophores in order of price, the processing unit 101 may have price information of the fluorophore or the fluorophore-labeled biomolecule (for example, fluorochrome-labeled antibody). The price information may be associated with each fluorophore or each fluorophore-labeled biomolecule. The information processing device may have the price information in advance, or the information processing device may acquire the price information when the use history information acquisition button 408 or the document information acquisition button 412 described above is selected. For example, the information processing device can acquire the price information from a predetermined database or via a predetermined network when these buttons are selected.

### (3-2-7) Use of Stock Information or Price Information

In the embodiment, the information processing device according to the present disclosure may present a fluorophore candidate on the basis of stock information. For the presentation, the storage unit 102 may be configured to store stock information.

For example, the processing unit 101 can display a window 430 as illustrated in FIG. 8P. The window is the same as the window 420 described above except that the window has an input box 431 that prompts selection for use of the stock information. In FIG. 8P, the input box is checked. In a case where the input box is checked, the processing unit 101 excludes, for example, the fluorophore out of stock from the fluorophore use history list and/or the in-document use result list. Thus, the fluorophore out of stock is excluded from the candidates.

In another embodiment, the information processing device according to the present disclosure can present a fluorophore candidate on the basis of price information. For the presentation, the storage unit 102 may be configured to store price information.

For example, the processing unit 101 can display a window 440 as illustrated in FIG. 8O. The window is the same as the window 430 described above except that the window has an input box 441 that prompts selection for use of the price information. In FIG. 8P, the input box is checked. In a case where the input box is checked, the processing unit 101 arranges, for example, the fluorophore use history list and/or the in-document use result list in ascending order of price. Thus, it is easy to select a fluorophore candidate with low cost for each biomolecule.

### (3-2-8) Search for Biomolecules

In the embodiment, the processing unit may be configured to be able to perform search for a biomolecule to be analyzed in biological sample analysis. The processing unit can perform the search on the fluorophore use history information, the in-document fluorophore use result information, or both of them on the basis of the input keyword.

For example, the processing unit 101 can display a window 450 as illustrated in FIG. 8R. The window is the same as the window 400 described above except that the window has a search button group 451. In FIG. 8R, search buttons are respectively arranged beside name input boxes in the biomolecule input area 402. When the user inputs a keyword in the name input box and selects a search button, the processing unit 101 performs search for the fluorophore use history information and/or the in-document fluorophore use result information by using the keyword. Then, the processing unit 101 can display the hit biomolecule in the name input box or in another window. In the search, the processing unit 101 may execute search processing such that a biomolecule including a keyword similar to the keyword is hit. Thus, it is possible to cope with notation variability.

### (3-3) Modification Example of Generation Processing of Combination List of Antibodies and Fluorochromes

The information processing device according to the present disclosure may perform presentation processing of a fluorophore candidate by a predetermined algorithm in addition to the presentation of the fluorophore candidate based on the fluorophore use history information, the in-document fluorophore use result information, or both of them, which is described in (3-2-3) above. That is, the processing unit 101 may further specify the combination of the biomolecule and the fluorophore in addition to the combination of the biomolecule selected in (3-2-3) described above and the fluorophore selected from the fluorophore candidates. Then, the combination list generated by the processing unit 101 may include a combination of the biomolecule and the fluorophore, which is specified by the processing unit, in addition to the combination of the selected biomolecule and the fluorophore selected from the fluorophore candidates.

Thus, for example, it is possible to present a fluorophore combined with a biomolecule that is not included in any of the fluorophore use history information and the in-document fluorophore use result information.

Furthermore, for example, it is possible to cope with a case where the user does not desire to use the fluorophore candidate presented on the basis of the fluorophore use history information or the in-document fluorophore use result information.

Hereinafter, the processing of generating a combination list of fluorophores for biomolecules, which uses the fluorophore use history information and/or the in-document fluorophore use result information, will be described.

(3-3-1) Example of Processing (Example of Processing in which Fluorophore Determined on basis of Use History Information or In-Document Result Information is Not Considered in Processing by Algorithm)

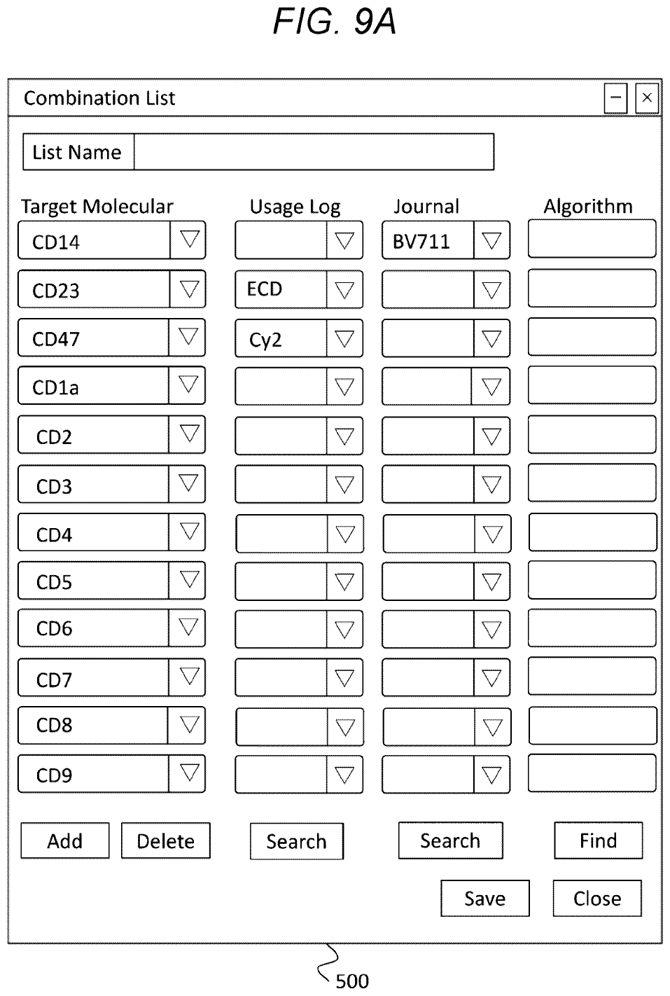

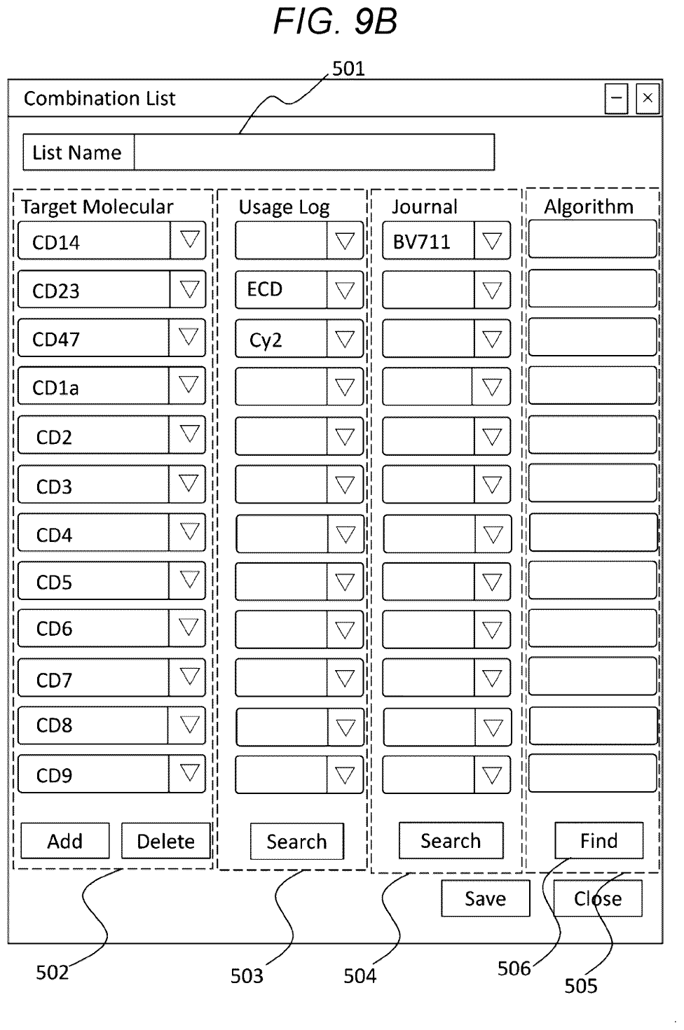

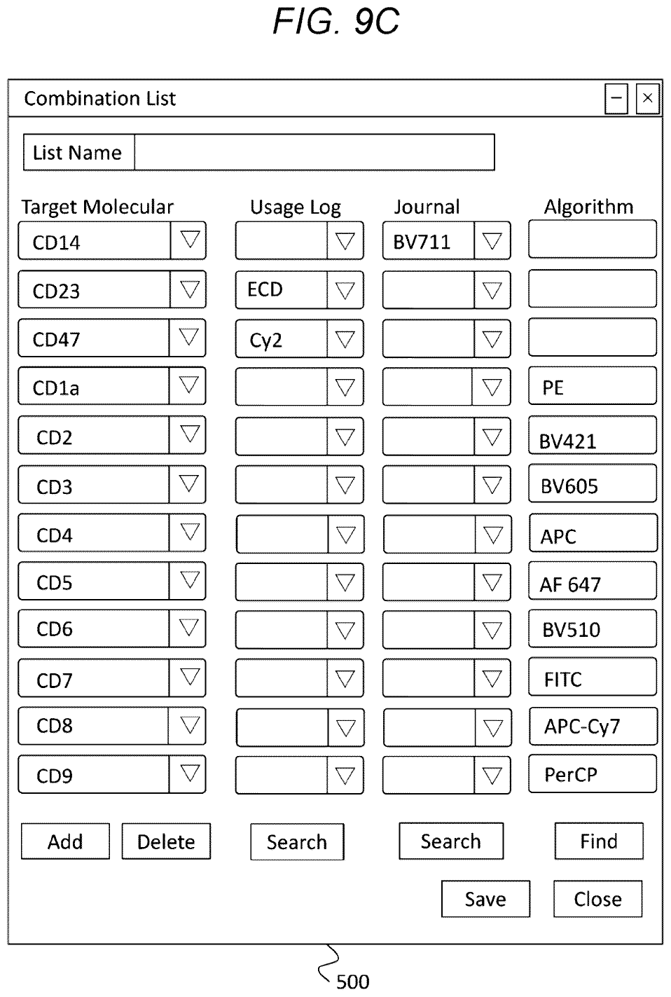

FIG. 9A illustrates an example of a window for receiving an input of biomolecules to be analyzed (for example, antigens). As illustrated in FIG. 9B, a window 500 illustrated in FIG. 9A includes a list name input area 501 to which the name of the combination list is input, a biomolecule input area 502 to which the analysis target biomolecule is input, a use information display area 503 in which the fluorophore use history information is displayed and the fluorophore is selected, and a document information display area 504 in which the in-document fluorophore use result information is displayed and the fluorophore is selected. Moreover, the window 500 includes an area 505 in which the fluorophore assigned to each biomolecule by the algorithm is displayed. In the area 505, the fluorophore selected by executing the following processing flow as the algorithm is displayed.

The list name input area 501 is the same as the area 401 described in (3-2-3) above. The biomolecule input area 502, the use information display area 503, and the document information display area 504 are the same as the area 402, the use information display area 403, and the document information display area 404, which are described in (3-2-3) above except that the number of displayed boxes is different.

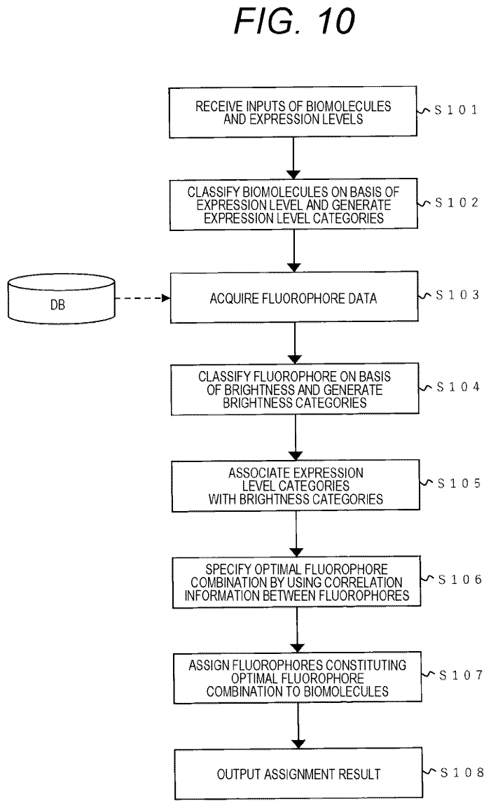

As illustrated in FIG. 9A, in the biomolecule input area, CD14, CD23, CD47, CDla, CD2, CD3, CD4, CD5, CD6, CD7, CD8, and CD9 are input. It is assumed the user desires to assign a fluorophore to each of these 11 biomolecules. Among these biomolecules, for CD14, CD23, and CD47, BV711, ECD, and Cy2 are respectively assigned by the processing described in (3-2-3) above. On the other hand, the fluorophore may be assigned to CDla, CD2, CD3, CD4, CD5, CD6, CD7, CD8, and CD9, for example, by the processing unit executing the processing to be described below. As illustrated in FIG. 9B, the window 500 may include, for example, a button 506 (Find button) for executing the processing, and the processing unit 100 starts execution of the processing when the button is selected. The processing will be described below with reference to FIG. 10. FIG. 10 is a flowchart of the processing.

In step S101 of FIG. 10, the information processing device 100 (in particular, the input unit 103) receives inputs of a plurality of biomolecules and expression levels of the plurality of biomolecules.

The biomolecules may be antigens to be measured in flow cytometry (for example, surface antigens, cytokines, or the like), or may be antibodies that capture antigens to be measured. In a case where the plurality of biomolecules is antigens, the expression levels may be expression levels of the antigens. In a case where the plurality of biomolecules is antibodies, the expression levels may be expression levels of antigens captured by the antibodies.

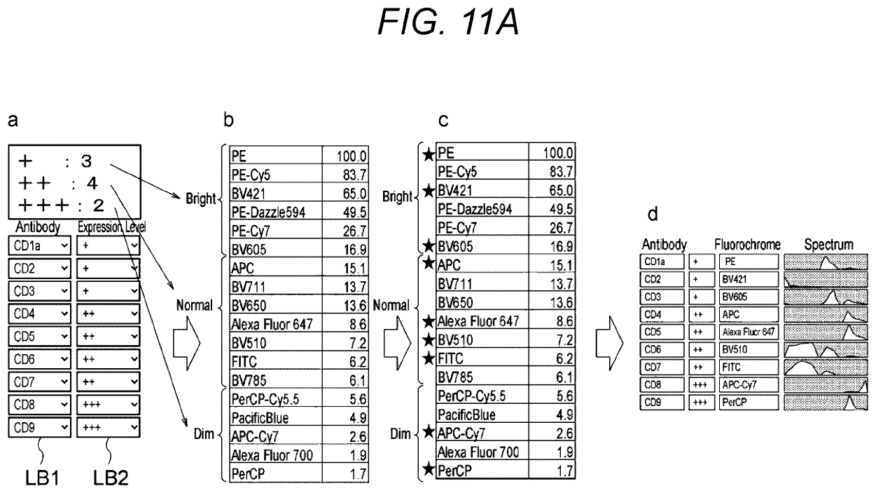

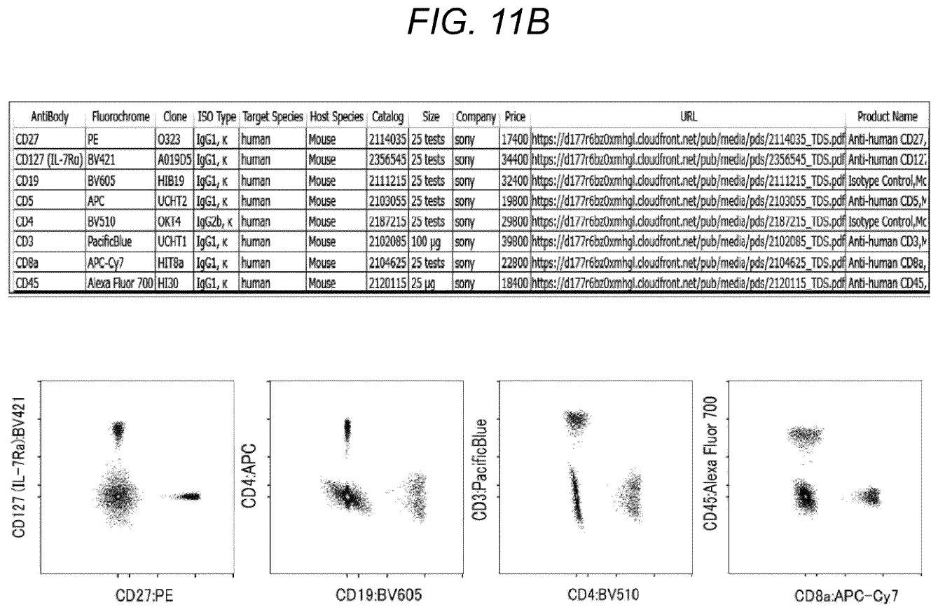

The processing unit 101 can cause the output unit 104 (particularly, a display device) to display an input reception window for receiving the input to prompt the user to perform the input. The input reception window may include, for example, a biomolecule input reception box and an expression level reception box such as an “Antibody” box and an “Expression Level” box illustrated in a of FIG. 11A.

The biomolecule input reception boxes may be, for example, a plurality of list boxes LB1 prompting selection of biomolecules, as illustrated in the “Antibody” box in a of FIG. 11A. In a of FIG. 11A, nine list boxes are described for convenience of description, but the number of list boxes is not limited thereto. The number of list boxes may be, for example, five to 300 or 10 to 200.

When the button 506 (Find button) is selected, the processing unit 101 may automatically display biomolecules (CDla, CD2, CD3, CD4, CD5, CD6, CD7, CD8, and CD9) to which neither the fluorophore use history information nor the in-document fluorophore use result information is assigned in each list box.

Alternatively, when the user enables each list box through an operation such as clicking or touching, the processing unit 101 may display a list of options of biomolecules above or below the list box. When the user selects one biomolecule from the list, the list is closed and the selected biomolecule is displayed.

In a of FIG. 11A, a screen after the input of the biomolecules is displayed. In response to the selection of antigens to be captured by antibodies, as illustrated in a of FIG. 11A, for example, “CD1a”, “CD2” or the like is displayed.

Furthermore, the expression level reception boxes may be, for example, a plurality of list boxes LB2 prompting selection of an expression level, as illustrated in an “Expression Level” field in a of FIG. 11A. The number of list boxes LB2 prompting the selection of the expression level may be the same as the number of list boxes LB1 prompting the selection of the biomolecule. In a of FIG. 11A, nine list boxes are described for convenience of description, but the number of list boxes is not limited thereto. The number of list boxes may be, for example, five to 300 or 10 to 200.

When the user enables each list box through an operation such as clicking or touching, the processing unit 101 displays a list of options of expression levels above or below the list box. When the user selects one biomolecule from the list, the list is closed and the selected expression level is displayed.

In a of FIG. 11A, a screen after selection of expression levels by the user is displayed. In response to selection of the levels of the expression levels, as illustrated in a of FIG. 11A, for example, “+”, “++”, and “+++” are displayed. In a of FIG. 11A, for example, “+” is selected as the expression level of the biomolecule “CD1a”. Furthermore, “++” is selected as the expression level of the biomolecule “CD4”. The symbols “+”, “++”, and “+++” mean that the expression level increases in this order. In the present description, the “expression level” may mean, for example, the level of the expression level, or may be a specific numerical value of the expression level. Preferably, as illustrated in a of FIG. 11A, the expression level means the level of the expression level. The level of the expression level may be preferably divided into two to 20 steps, more preferably divided into two to 15 steps, and still more preferably divided into two to 10 steps, and may be divided into, for example, three to 10 steps.

After the selection of the biomolecules and the expression levels is completed as described above, for example, when the user clicks a selection completion button (not illustrated) in the input reception window, the processing unit 101 receives the input of the selected biomolecules and the expression levels.

In step S102, the processing unit 101 classifies a plurality of biomolecules selected in step S101 on the basis of the expression level selected for each biomolecule, and generates one or a plurality of expression level categories, in particular, a plurality of expression level categories. The number of expression level categories may be, for example, a value corresponding to the number of expression level levels, and may be preferably two or more, and more preferably three or more. The number of expression level categories may be preferably two to 20, preferably three to 15, and still more preferably three to 10.

In a of FIG. 11A, the expression level “+”, “++”, or “+++” is selected for each of the plurality of biomolecules. The processing unit 101 classifies the biomolecules whose selected expression level is “+” into an expression level category “+”. Similarly, the processing unit 101 classifies biomolecules whose selected expression level is “++” or “+++” into an expression level category “++” or an expression level category “+++”, respectively. In this manner, the processing unit 101 generates three expression level categories. Each expression level category includes biomolecules whose corresponding expression level is selected. In a of FIG. 11A, three biomolecules at the expression level “+”, four biomolecules at the expression level “++”, and two biomolecules at the expression level “+++” are input.

In step S103, the processing unit 101 acquires a list of fluorophores capable of labeling the biomolecules input in step S101. The list of the fluorophores may be acquired, for example, from a database existing outside the information processing device 100 via the communication unit 105, or may be acquired from a database stored inside the information processing device 100 (for example, the storage unit 102).

The list for the fluorophores may include, for example, a name and brightness for each fluorophore. Furthermore, the list for the fluorophores preferably also includes the fluorescence spectrum of each fluorophore. The fluorescence spectrum of each fluorophore may be acquired from the database as data different from the list.

Preferably, the list may selectively include fluorophores usable in a device (for example, a microparticle analyzer) in which the sample is analyzed using a combination of biomolecules and fluorophores. Since the fluorophores unusable in the device are deleted from the list, it is possible to reduce the burden on the device in the processing to be described later (in particular, the calculation processing of the correlation information).

In step S104, the processing unit 101 classifies the fluorophores included in the list regarding the fluorophores acquired in step S103 on the basis of the brightness of each fluorophore, and generates one or a plurality of brightness categories, in particular, a plurality of brightness categories.

In step S104, preferably, the processing unit 101 generates the brightness categories with reference to the expression level categories generated in step S102. Thus, it is possible to more efficiently associate the brightness categories to be generated with the expression level categories and generate the combination of the biomolecules and the fluorophores. Specific content of the reference will be described below.

The classification based on the brightness may be classification based on a fluorescence amount or a fluorescence intensity. In order to perform the classification, for example, a numerical range of the fluorescence amount or the fluorescence intensity may be associated with each of the brightness categories. Then, the processing unit 101 can classify each of the fluorophores included in the list into a brightness category associated with the numerical range including the fluorescence amount or the fluorescence intensity with reference to the fluorescence amount or the fluorescence intensity of each fluorophore.

Preferably, in step S104, the processing unit 101 generates brightness categories with reference to the number of expression level categories generated in step S102. Particularly preferably, in step S104, the processing unit 101 generates the same number of brightness categories as the number of expression level categories generated in step S102. Therefore, the expression level categories and the brightness categories can be associated on a one-to-one basis. In addition, it is possible to prevent generation of fluorophores that are not considered in generation of a combination list to be described later, and it is possible to generate a better combination. The number of brightness categories may be, for example, a value corresponding to the number of expression level categories, and may be preferably two or more, and more preferably three or more. The number of expression level categories may be preferably two to 20, preferably three to 15, and still more preferably three to 10. For example, as illustrated in b of FIG. 11A, three brightness categories (Bright, Normal, and Dim) may be generated. In these three brightness categories, the brightness decreases in this order, that is, all the fluorophores included in Bright are brighter than any of the fluorophores included in Normal, and all the fluorophores included in Normal are brighter than any of the fluorophores included in Dim.

Preferably, in step S104, the processing unit 101 generates brightness categories with reference to the number of biomolecules included in each of the expression level categories generated in step S102. Particularly preferably, in step S104, the processing unit 101 classifies the fluorophores into the brightness categories such that the fluorophores equal to or more than the number of biomolecules included in the expression level categories generated in step S102 are included in the associated brightness categories. Thus, it is possible to prevent generation of biomolecules to which fluorophores is not assigned in generation of a combination list described later.

In step S105, the processing unit 101 associates the expression level categories generated in step S102 with the brightness categories generated in step S104. Preferably, the processing unit 101 associates one expression level category with one brightness category. Furthermore, the processing unit 101 can perform association such that the expression level category and the brightness category correspond on a one-to-one basis. That is, the association can be performed such that two or more expression level categories are not associated with one brightness category.

In a particularly preferred embodiment of the present disclosure, the processing unit 101 can perform the association such that an expression level category with a smaller expression level is associated with a brighter brightness category. For example, the processing unit 101 associates the expression level category having the smallest expression level with the brightness category having the brightest brightness, and then associates the expression level category having the second smallest expression level with the brightness category having the second brightest brightness, and similarly, this association can be repeated until there are no more expression level categories. Conversely, the processing unit 101 associates the expression level category having the highest expression level with the brightness category having the lowest brightness, and then associates the expression level category having the second highest expression level with the brightness category having the second lowest brightness, and similarly, this association can be repeated until there are no more expression level categories.

In this embodiment, for example, as indicated by arrows between a and b in FIG. 11A, the processing unit 101 associates the expression level categories “+”, “++”, and “+++” with the brightness categories “Bright”, “Normal”, and “Dim”, respectively.

As described above, the expression level categories generated in the present disclosure may be associated with the brightness categories such that, preferably, an expression level category in which biomolecules exhibiting a smaller expression level are classified corresponds to a brightness category in which brighter fluorophores are classified.

In step S106, the processing unit 101 specifies an optimal fluorophore combination by using correlation information between fluorophores. The optimal fluorophore combination may be, for example, a fluorophore combination that is optimal from the viewpoint of the correlation between the fluorescence spectra, may be more particularly a fluorophore combination that is optimal from the viewpoint of the correlation coefficient between the fluorescence spectra, and may be even more particularly a fluorophore combination that is optimal from the viewpoint of the square of the correlation coefficient between the fluorescence spectra. The correlation coefficient may be, for example, any of a Pearson correlation coefficient, a Spearman correlation coefficient, or a Kendall correlation coefficient, and is preferably a Pearson correlation coefficient.

The correlation information between the fluorophores may be preferably correlation information between fluorescence spectra. That is, in one preferred embodiment of the present disclosure, the processing unit 101 specifies an optimal fluorophore combination by using the correlation information between fluorescence spectra.

For example, the Pearson correlation coefficient can be calculated between two of fluorescence spectra X and Y as below.

First, for example, the fluorescence spectra X and Y can be expressed as below.

Fluorescence spectrum X=(X₁, X₂, . . . , X₃₂₀), mean=μₓ, standard deviation=σₓ(where X₁ to X₃₂₀ are fluorescence intensities at 320 different wavelengths, the mean μₓ is a mean of these fluorescence intensities, and the standard deviation σₓ is a standard deviation of these fluorescence intensities)

Fluorescence spectrum Y=(Y₁, Y₂, . . . , Y₃₂₀), mean=μ_{y}, standard deviation=σ_{y}(where Y₁ to Y₃₂₀ are fluorescence intensities at 320 different wavelengths, the mean μ_{y} is a mean of these fluorescence intensities, the standard deviation ox is a standard deviation of these fluorescence intensities)

Note that the numerical value “320” is a value set for convenience of description, and the numerical value used in the calculation of the correlation coefficient is not limited thereto. The numerical value may be appropriately changed according to the configuration of the fluorescence detector, for example, the number of PMTs (photomultiplier tubes) used for fluorescence detection and the like.

The Pearson correlation coefficient R between the fluorescence spectra X and Y is obtained by Equation 1 below.

$$R = \frac{\sum {Z}_{Xn} {Z}_{Yn}}{N} \qquad [ Math . 1 ]$$

In Equation of Math. 1, Z_{Xn} (n is 1 to 320) is a standardized fluorescence intensity and is expressed as below.

$$z \times 1 = ( {x}_{1} - {μ}_{x} ) \div {σ}_{x} , Z \times 2 = ( {X}_{2} - {μ}_{x} ) \div {σ}_{x} , … Z \times 320 = ( {X}_{320} - {μ}_{x} ) \div {σ}_{x}$$

Similarly, Z_{Yn} (n is one to 320) is also expressed as below.

$$Zy 1 = ( {Y}_{1} - {μ}_{y} ) \div {σ}_{y} , Zy 2 = ( {Y}_{2} - {μ}_{y} ) \div {σ}_{y} , … Zy 320 = ( {Y}_{320} - {μ}_{y} ) \div {σ}_{y}$$

Furthermore, in Equation of Math. 1, N is the number of data.

An example of how to specify the optimal fluorophore combination will be described below.

The processing unit 101 selects the same number of fluorophores as the “number of biomolecules belonging to an expression level category associated with a certain brightness category” from the certain brightness category. The selection of fluorophores is performed for all brightness categories. Thus, the same number of fluorophores as the “number of a plurality of biomolecules used for analysis of a sample” are selected, and in this manner, one fluorophore combination candidate is obtained.

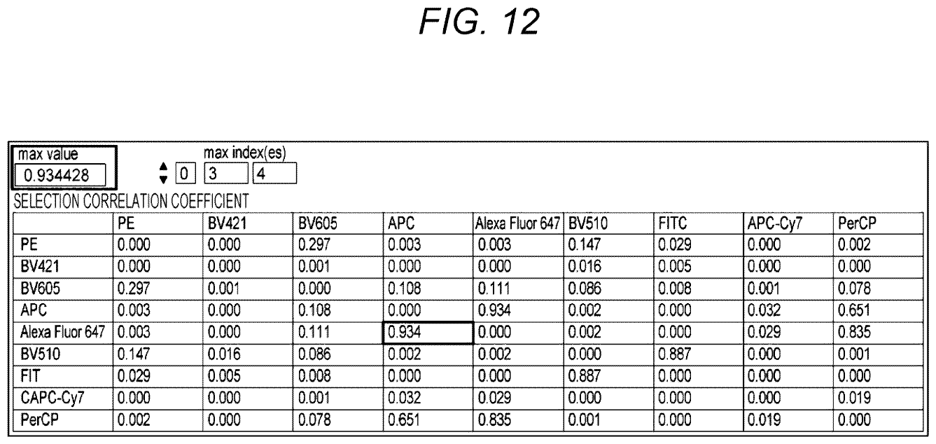

Next, the processing unit 101 calculates the square of the correlation coefficient (for example, Pearson correlation coefficient) between the fluorescence spectra for a combination of any two fluorophores included in the fluorophore combination candidate. The processing unit 101 calculates the square of the correlation coefficient for all combinations. For example, through the calculation processing, the processing unit 101 obtains a matrix of correlation coefficient square values as illustrated in FIG. 12. Then, the processing unit 101 specifies the maximum correlation coefficient square value from the matrix of correlation coefficient square values. For example, in FIG. 12, the correlation coefficient between the fluorescence spectrum of Alexa Fluor 647 and the fluorescence spectrum of APC is 0.934, and the processing unit 101 specifies this value as the maximum correlation coefficient square value (a portion surrounded by a square in the upper left of the FIG. 12).

Note that the smaller the correlation coefficient square value is, the less similar the two fluorophore spectra are. That is, the two fluorophores having the maximum correlation coefficient square value can mean the two fluorophores having the most similar fluorescence spectra among the fluorophores included in the fluorophore combination candidate.

By the above-described processing, the processing unit 101 specifies the maximum correlation coefficient square value for one fluorophore combination candidate.

Here, in a case where the “number of fluorophores belonging to a certain brightness category” is larger than the “number of biomolecules belonging to an expression level category associated with the certain brightness category”, there is a plurality of combinations of fluorophores selected from the certain brightness category. For example, there are six kinds of fluorophore combinations (=₄C₂) in a case where two fluorophores are selected from four fluorophores. Therefore, for example, in a case where there are three brightness categories, four fluorophores belong to any of the three brightness categories, and two fluorophores are selected from each brightness category, there are 216 fluorophore combination candidates of 6×6×6.

In the present disclosure, the processing unit 101 specifies the maximum correlation coefficient square value as described above for all possible fluorophore combination candidates. For example, in a case where there are 216 fluorophore combination candidates, the processing unit 101 specifies the maximum correlation coefficient square value of each of the 216 fluorophore combination candidates. Then, the processing unit 101 specifies a fluorophore combination candidate having the smallest specified maximum correlation coefficient square value. The processing unit 101 specifies the fluorophore combination candidate specified in this manner as an optimal fluorophore combination.

c of FIG. 11A illustrates the specifying result of the optimal fluorophore combination. In c of FIG. 11A, the fluorophores constituting the specified optimal fluorophore combination are marked with stars.

Note that, in a case where there are two or more fluorophore combination candidates having the smallest maximum correlation coefficient square value, the processing unit 101 can compare the second largest correlation coefficient square value with respect to the two or more fluorophore combination candidates and specify a fluorophore combination candidate having the second largest correlation coefficient square value smaller as the optimal fluorophore combination. In a case where the second largest correlation coefficient square values are the same, the third largest correlation coefficient square values can be compared.

In the above description, the maximum correlation coefficient square value is referred to in order to specify the optimal fluorophore combination, but what is referred to in order to specify the optimal fluorophore combination is not limited thereto. For example, it may be an average value or a total value from the largest value to the nth (here, n may be any positive number, for example, two to 10, particularly two to eight, and more particularly two to five) largest value among the correlation coefficient square values. The processing unit 101 may specify the fluorophore combination candidate having the smallest average value or the smallest total value as the optimal fluorophore combination.

In step S107, the processing unit 101 assigns the fluorophores constituting the optimal fluorophore combination specified in step S106 to the plurality of biomolecules. More specifically, the processing unit 101 assigns each of the fluorophores constituting the optimal fluorophore combination to the biomolecule belonging to the expression level category associated with the brightness category to which the fluorophore belongs.

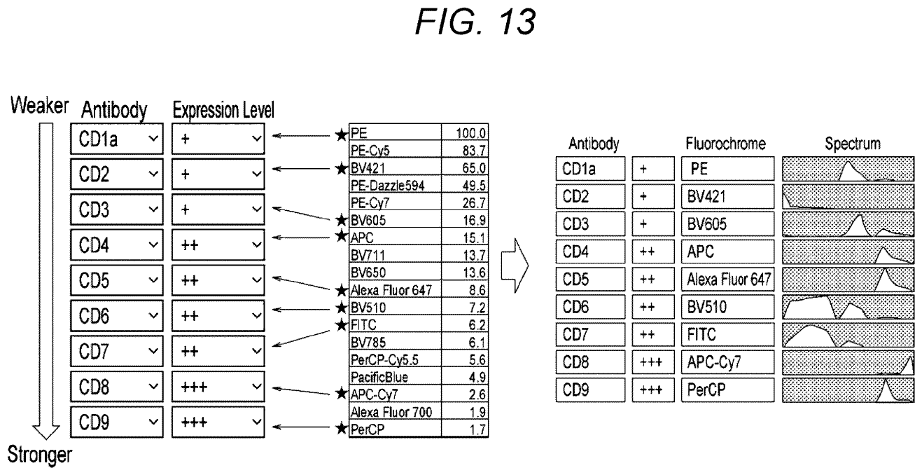

In a case where two or more fluorophores are included in one brightness category, two or more biomolecules may be included in the associated expression level category. In this case, a fluorophore having a brighter brightness may be assigned to a biomolecule having a lower expression level (or expected to have a lower expression level). FIG. 13 illustrates a conceptual diagram related to such assignment.

The processing unit 101 generates a combination of a fluorophore and a biomolecule for each biomolecule by the assignment processing described above. In this manner, the processing unit 101 generates a combination list of fluorophores for biomolecules.

An example of a generation result of the combination list is illustrated in d of FIG. 11A. The processing unit 101 may output the generation result of the combination list. In this case, for example, as in d of FIG. 11A, in addition to the combination of the biomolecule and the fluorophore, the processing unit 101 may display the fluorescence spectrum of each fluorochrome. By displaying the fluorescence spectrum, it is possible for the user to visually confirm whether there is spectrum overlap.

When a predetermined button (not illustrated) is selected after the combination list is generated in this manner, the processing unit 101 displays the fluorophores included in the combination list in the window illustrated in FIG. 9B. Thus, for example, as illustrated in FIG. 9C, the fluorophore is assigned to each biomolecule.

In step S108, the processing unit 101 can cause, for example, the output unit 104 to output the combination list generated in step S107. For example, the combination list can be displayed on the display device.

In step S108, the processing unit 101 can further display reagent information corresponding to the combination of the antibody (or antigen) and the fluorochrome on the output unit 104. The reagent information may include, for example, names of reagents, product numbers, manufacturer names, and prices. In order to display the reagent information, for example, the processing unit 101 may acquire the reagent information from a database existing outside the information processing device 100 or from a database stored inside the information processing device 100 (for example, the storage unit 102).

In step S108, the processing unit 101 may further display a simulation result (for example, various plots) regarding the separability in a case where the generated combination list is used. The processing unit 101 may further display separation performance expected in a case where the generated combination list is used.

FIG. 11B illustrates an example of the output result. In this example, simulation results are also illustrated in addition to the names of the antibodies (or antigens), the names of the fluorochromes, the names of the reagents, the product numbers, the manufacturer names, the prices, and the like.

By the above-described processing, the combination of the biomolecules and the fluorophores can be optimized, and the optimized combination list can be presented to the user.

### (3-3-2) Another Example of Processing (Example of Fluorescence Spectrum Used in Calculation of Correlation Coefficient)

As described in (3-3-1) above, the processing unit 101 can acquire the fluorescence spectrum of each fluorophore in step S103, and then can specify the optimum fluorophore combination by using the correlation information between the fluorescence spectra in step S106. The fluorescence spectrum used to acquire the correlation information may have a horizontal axis indicating a wavelength or a photodetector number corresponding to the wavelength, and a vertical axis indicating fluorescence intensity, in particular, fluorescence intensity standardized by a fluorescence intensity maximum value.

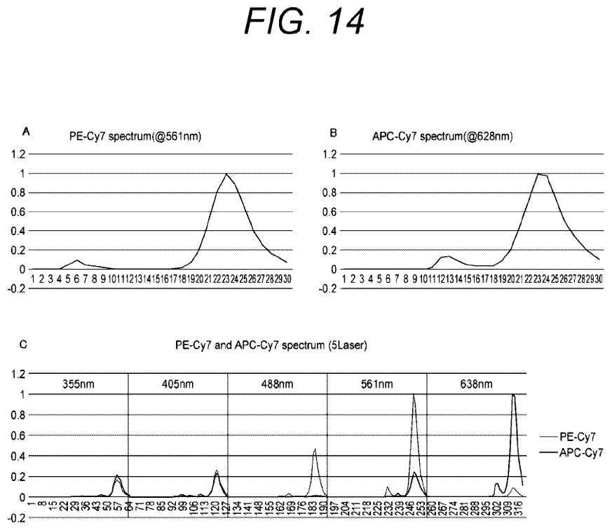

The fluorescence spectrum used in the present disclosure may be a fluorescence spectrum of fluorescence generated in a case where the fluorophore is irradiated with excitation light having one wavelength, or may be a combination of a plurality of fluorescence spectra obtained in a case where the fluorophore is irradiated with excitation light having two or more different wavelengths, in particular, a combination of two or more fluorescence spectra. These embodiments will be described below with reference to FIG. 14.

A and B of FIG. 14 illustrate examples of fluorescence spectra of fluorescence generated in a case where the fluorophores are irradiated with excitation light having one wavelength. A of FIG. 14 is a fluorescence spectrum of fluorescence obtained in a case where PE-Cy5 is irradiated with an excitation laser beam of 561 nm. The horizontal axis of the fluorescence spectrum indicates the photodetector number corresponding to each wavelength, that is, 30 photodetectors are used to obtain the fluorescence spectrum. The vertical axis of the fluorescence spectrum is the fluorescence intensity standardized by the fluorescence intensity maximum value. B of FIG. 14 is a fluorescence spectrum of fluorescence obtained in a case where APC-Cy5 is irradiated with an excitation laser beam of 628 nm. The vertical axis and the horizontal axis of the fluorescence spectrum are the same as those in A of FIG. 14.

In the present disclosure, the fluorescence spectrum as illustrated in A or B of FIG. 14 may be used. However, the fluorescence spectra in A and B of FIG. 14 are also shown to have similar spectral waveforms, and two fluorophores may not be distinguishable. Then, in the present disclosure, by using a combination of a plurality of fluorescence spectra obtained in a case where a fluorophore is irradiated with excitation light having a plurality of different wavelengths, in particular, a combination of a plurality of fluorescence spectra, it is possible to more reliably distinguish between two fluorophores.

C of FIG. 14 illustrates an example of data obtained by combining a plurality of fluorescence spectra obtained in a case where the fluorophore is irradiated with excitation light having a plurality of different wavelengths. The data illustrated in C of FIG. 14 is data obtained by combining fluorescence spectra of five fluorescences generated in a case where excitation laser beams having five different wavelengths are emitted. The five different wavelengths are 355 nm, 405 nm, 488 nm, 561 nm, and 638 nm. All the horizontal axes of five fluorescence spectra combined to obtain the data indicate the photodetector numbers corresponding to each wavelength, that is, 64 photodetectors are used to obtain the fluorescence spectrum. Furthermore, the vertical axes of the five fluorescence spectra indicate the fluorescence intensities, and the five fluorescence spectra are standardized with the maximum value of the fluorescence intensities of the five fluorescence spectra as one.

In C of FIG. 14, a thin line is fluorescence spectrum coupled data of PE-Cy5, and a thin line is fluorescence spectrum coupled data of APC-Cy5. When these two coupled data are compared, it can be seen that the waveforms of the fluorescence spectra obtained in a case where excitation light beams of, for example, 488 nm, 561 nm, and 638 nm are emitted are completely different. Therefore, the fluorescence of two fluorochromes can be more reliably distinguished by these two coupled data.

As described above, in the present disclosure, the processing unit 101 can easily specify an optimal fluorophore combination by using combination data of a plurality of fluorescence spectra, in particular, by using coupled data of a plurality of fluorescence spectra, in order to obtain the correlation information. The combination data and the coupled data may be, for example, data obtained by performing predetermined standardization processing on a plurality of fluorescence spectra as described above.

Furthermore, by obtaining the correlation information using the fluorescence spectrum as described above, the same processing flow can be applied to various microparticle analyzers having different optical systems.

(3-3-3) Still Another example of Processing (Example of Executing Separability Evaluation)

In a preferred embodiment of the present disclosure, the processing unit 101 can evaluate the separability for the generated combination list. For example, the processing unit 101 can generate simulation data regarding the generated combination list and evaluate the separability regarding the combination list by using the simulation data. By performing the evaluation of the separability, the accuracy of optimization can be enhanced.

For example, by performing the evaluation of the separability, it is possible to confirm whether the combination list generated in step S107 exhibits desired separation performance, or it is also possible to generate a combination list exhibiting better separation performance according to the confirmation result.

In this embodiment, for example, the processing unit 101 can further generate a modified combination list in which at least one fluorophore of a set of fluorophores included in the combination list is changed to another fluorophore according to the evaluation result of the separability, and can further perform the separability evaluation related to the modified combination list. By generating the modified combination list and then performing the separability evaluation, a combination list that exhibits better separation performance can be generated.

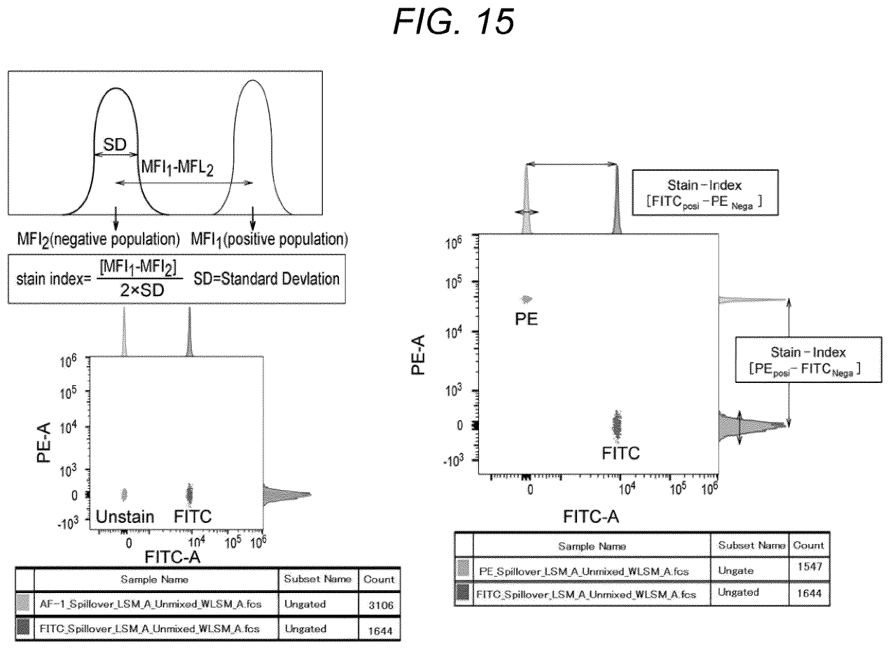

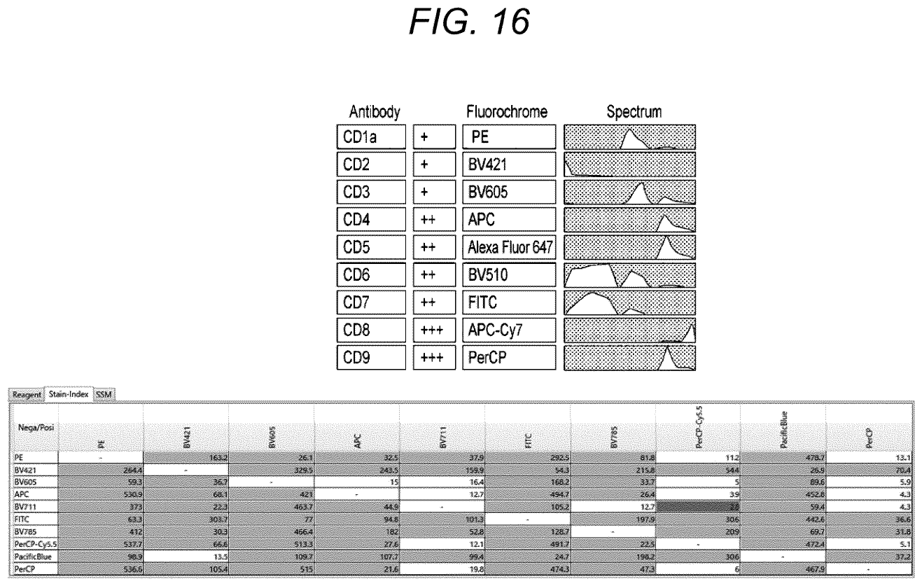

The evaluation of the separability may be, for example, the evaluation using a stain index, and more preferably the evaluation using an inter-fluorophore stain index. In the technical field, the stain index is an index indicating the performance of a fluorophore (fluorochrome) itself, and is defined by the fluorescence amount of stained particles and unstained particles and the standard deviation of unstained particle data, for example, as illustrated in the left of FIG. 15. The unstained particle data replaced with particles stained with another fluorophore is an inter-fluorophore stain index, for example, as illustrated in the right of FIG. 15. By using the inter-fluorophore stain index, it is possible to evaluate the separation performance between the fluorophores in consideration of the leakage amount due to the overlapping of the fluorescence spectra, the fluorescence amount, and noise. FIG. 16 illustrates an example of a result of calculating the inter-fluorophore stain index for all combinations of two fluorophores in the fluorophore group constituting the generated combination list.

Note that the processing unit 101 of the present disclosure can cause the output unit 104 to output the calculation result of all the combinations of two fluorophores in the fluorophore group constituting the combination list generated by the processing unit. Thus, the user can easily evaluate the separation performance.

For example, in the table of the inter-fluorochrome stain index as illustrated in FIG. 16, the separation performance is better as the number of regions having a smaller numerical value of the inter-fluorochrome stain index is smaller. In the present disclosure, first, a combination list may be generated on the basis of the expression level category, the brightness category, and the correlation information, and then, for example, the separability evaluation using an index such as a stain index may be performed. For example, in the generated combination list, a fluorophore combination having poor separation performance can be known through the separability evaluation, and a panel having better separation performance can be designed by changing the fluorophore combination.

Furthermore, when the panel design is performed by generating a combination list and performing separability evaluation (and panel modification as necessary) based on the above-described categories and the like, the calculation time can be reduced much more than when the panel design is performed by performing separability evaluation for every combination.

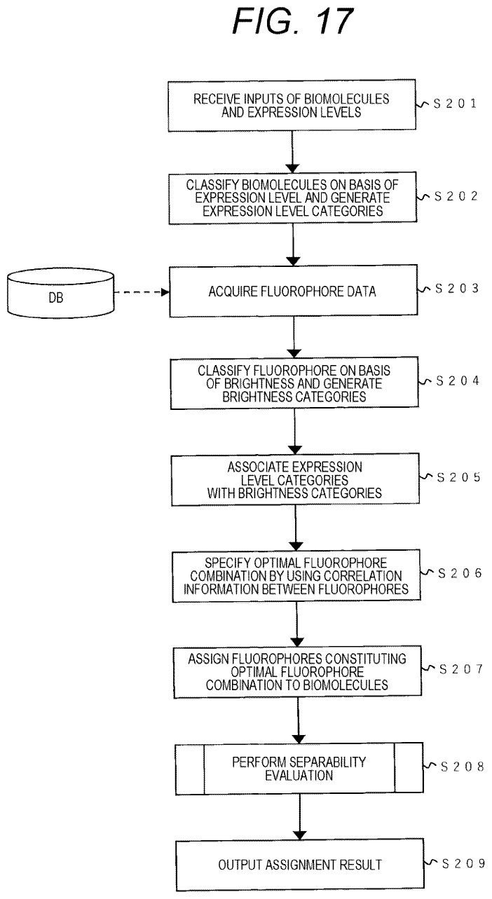

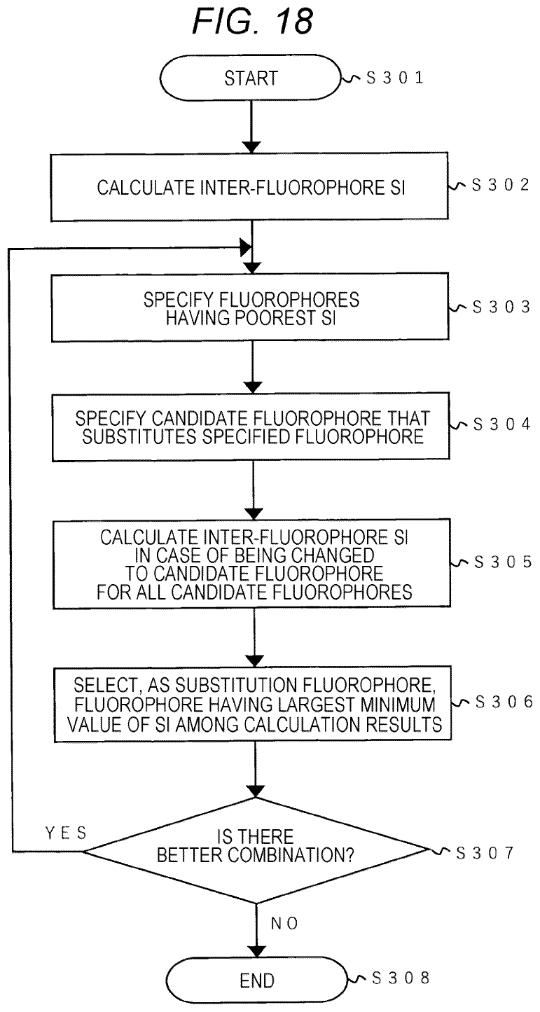

Hereinafter, an example of a processing flow in this embodiment will be described with reference to FIGS. 17 and 18. In the processing flow illustrated in FIG. 17, steps S201 to S207 and S209 are the same as steps S101 to S107 and S108 described with reference to FIG. 10, and the description thereof also applies to steps S201 to S207 and S209.

In step S208, the processing unit 101 evaluates the separability for the fluorophore group constituting the combination list generated by the assignment processing in step S207. An example of a more detailed processing flow of step S208 will be described with reference to FIG. 18.

In step S301 of FIG. 18, the processing unit 101 starts the separability evaluation processing.

In step S302, the processing unit 101 calculates an inter-fluorophore stain index (the stain index is also referred to as “SI” in the present description). The SI can be obtained using, for example, data obtained by generating simulation data using the combination list generated in step S207 and performing unmixing processing on the simulation data using spectral reference.

Here, the simulation data may be, for example, a data group obtained by being measured by a device (for example, a flow cytometer) on which analysis using reagents according to the combination list is performed. In a case where the device is a microparticle analyzer such as a flow cytometer, for example, the simulation data may be a data group obtained in a case where 100 to 1000 microparticles are actually measured. For the generation of the data group, for example, conditions such as noise of the device, staining variation, and the number of generated data may be considered.

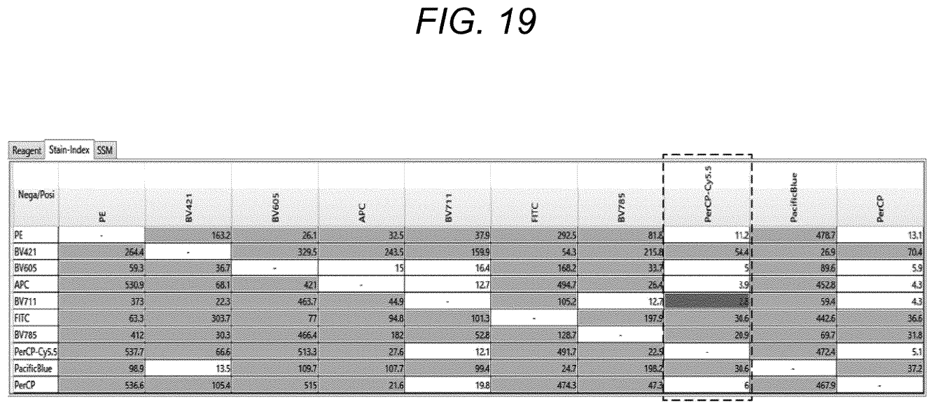

In step S302, for example, the processing unit 101 can acquire data of the inter-fluorophore SI as illustrated in FIG. 19. The data includes all of two different inter-fluorophore SIs in the fluorophore group constituting the combination list.

In step S303, the processing unit 101 specifies one or a plurality of fluorophores having poor separation performance, in particular, one fluorophore having poor separation performance, on the basis of the calculated inter-fluorophore SIs. For example, the processing unit 101 can specify, as one fluorophore having poor separation performance, a fluorophore treated as positive among two fluorophores for which the smallest inter-fluorophore SI is calculated.

For example, with respect to the inter-fluorophore SI data illustrated in FIG. 19, in step S303, the processing unit 101 specifies, as one fluorophore having poor separation performance, a fluorophore “PerCP-Cy5.5” treated as positive (posi) among the two fluorophores for which the smallest inter-fluorophore SI “2.8” is calculated.

In step S304, the processing unit 101 specifies a candidate fluorophore that substitutes the fluorophore having poor separation performance specified in step S303. For example, the candidate fluorophore may be specified as below. First, the processing unit 101 can refer to a brightness category to which the fluorophore having poor separation performance belongs, and specify a fluorophore not adopted in the combination list among fluorophores belonging to the brightness category as a candidate fluorophore. In addition, the processing unit 101 may select the candidate fluorophore from the brightness category having the closest brightness to the brightness category to which the fluorophore having poor separation performance belongs. The processing unit 101 can specify a fluorophore not adopted in the combination list among fluorophores belonging to the closest brightness category as a candidate fluorophore.

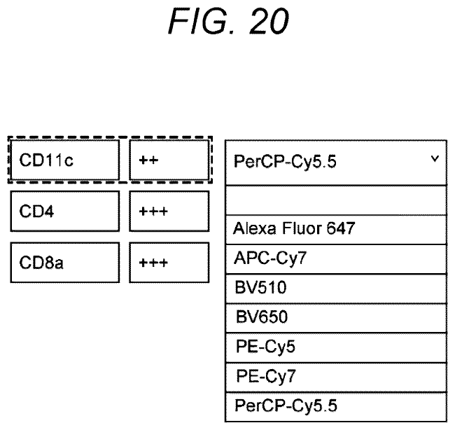

For example, in FIG. 20, the processing unit 101 specifies six fluorophores such as “Alexa Fluor 647” as candidate fluorophores to substitute the fluorophore “PerCP-Cy5.5” having poor separation performance. In this manner, a plurality of candidate fluorophores may be specified, or only one candidate fluorophore may be specified.

In step S305, the processing unit 101 calculates inter-fluorophore SI in a case where the fluorophore having poor separation performance specified in step S304 is changed to a candidate fluorophore. This calculation may be performed for all of the candidate fluorophores, respectively.

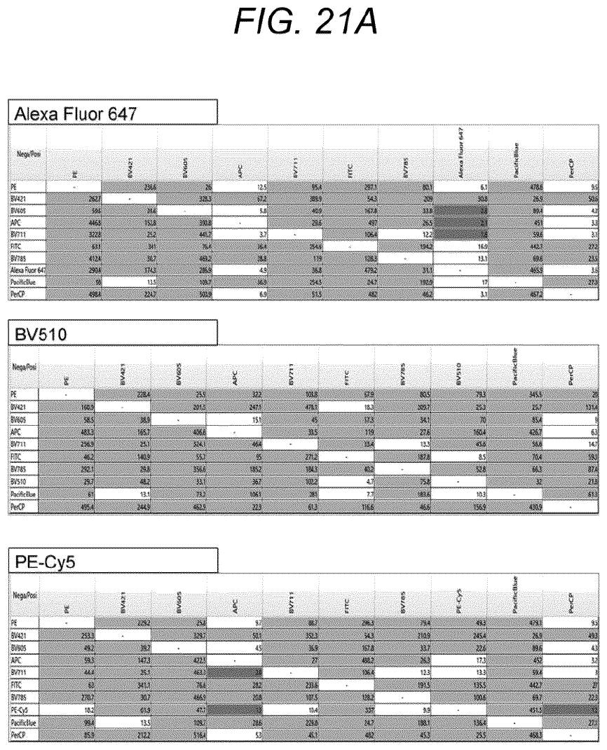

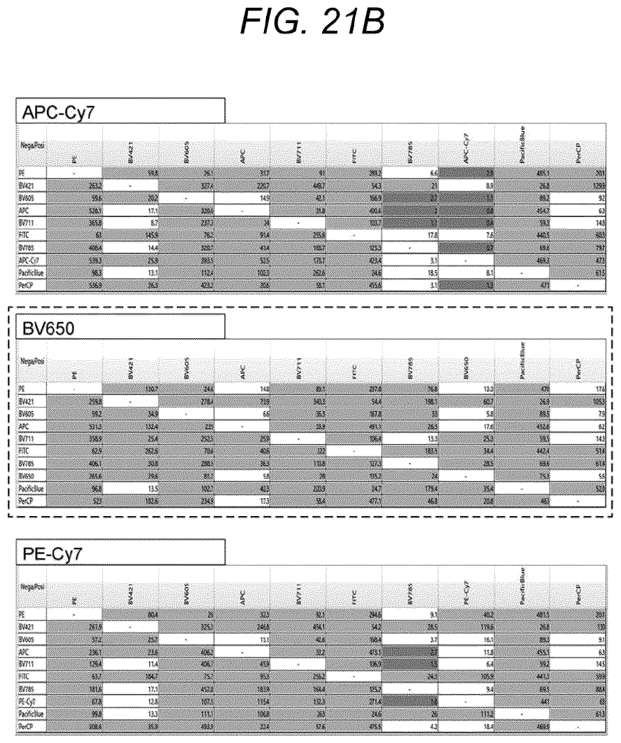

An example of the calculation result is illustrated in FIGS. 21A and 21B. In FIGS. 21A and 21B, for each of the six fluorophores described with reference to FIG. 20, the inter-fluorophore SI in a case where the fluorophore having poor separation performance is changed to the candidate fluorophore is illustrated.

In step S306, the processing unit 101 selects, as a fluorophore substituting the fluorophore having poor separation performance, a candidate fluorophore for which a calculation result having the largest minimum value of the inter-fluorophore SI is obtained among the calculation results in step S305.

For example, regarding the calculation results in FIGS. 21A and 21B, the minimum value of the inter-fluorophore SI related to “BV650” is the largest among the minimum values of the inter-fluorophore SIs in the calculation results of six candidate fluorophores. Thus, the processing unit 101 selects “BV650” as a fluorophore substituting “PerCP-Cy5.5”.

In step S307, the processing unit 101 determines whether there is a fluorophore combination that is better than the combination list obtained by substituting the fluorophore having poor separation performance with the fluorophore selected in step S306. For this determination, for example, step S303 to S306 may be repeated.

In a case where there is a combination in which the minimum value of the inter-fluorophore SI becomes larger as a result of repeating step S303 to S306, the processing unit 101 determines that there is a better fluorophore combination. In a case where the determination is made in this manner, the processing unit 101 returns the processing to step S303.

In a case where there is no combination in which the minimum value of the inter-fluorophore SI becomes larger as a result of repeating step S303 to S306, the processing unit 101 determines that there is no better fluorophore combination. In a case where it is determined that there is no better fluorophore combination, the processing unit 101 specifies a fluorophore combination in a stage immediately before repeating step S303 to S306 as an optimized combination list, and advances the processing to step S308.

In step S308, the processing unit 101 ends the separability evaluation processing and advances the processing to step S209.

Through the processing as described above, it is possible to present a combination list of biomolecules and fluorophores, which is optimized in consideration of separability.

### (3-3-4) Still Another Example of Processing (Processing Flow Considering Fluorophore Determined on Basis of Use History Information or In-Document Result Information in Processing by Algorithm)

In the processing flow based on the expression level and brightness described in (3-3-1) above, the biomolecule expression level and the fluorophore brightness related to the combination of the biomolecule and the fluorophore, which is determined using the fluorophore use history information and/or the in-document fluorophore use result information described in (3-2-3) above, are not considered. In the information processing device according to the present disclosure, in the processing flow, the biomolecule expression level and the fluorophore brightness related to the combination of the biomolecule and the phosphor, which is determined using the fluorophore use history information and/or the in-document fluorophore use result information, may be considered.

That is, in the embodiment of the present disclosure, the processing unit 101 may execute the processing flow based on the biomolecule expression level and the fluorophore brightness described in (3-3-1) above by using, as a fixed value, the combination of the biomolecule and the fluorophore, which is determined on the basis of the fluorophore use history information and/or the in-document fluorophore use result information.

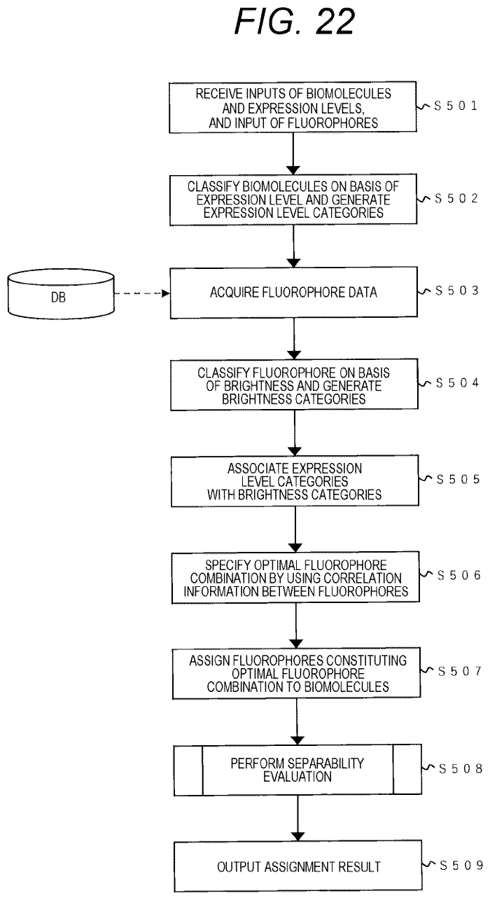

Hereinafter, the processing according to this embodiment will be described with reference to FIG. 22. FIG. 22 is a processing flow example according to this embodiment. In the processing flow illustrated in FIG. 22, steps S502 to S507 and S509 are the same as steps S102 to S107 and S109 described with reference to FIG. 10 except that the assigned fluorophore is specified in advance for some of the biomolecules, and the description thereof also applies to steps S102 to S107 and S109. Step S508 is the same as step S208 described with reference to FIG. 17 except that the assigned fluorophore is specified in advance for some of the biomolecules, and this description also applies to step S508.

In step S501 of FIG. 22, the information processing device 100 receives inputs of a plurality of biomolecules and expression levels of the plurality of biomolecules. Moreover, the input of the assigned fluorophore is received for some of the plurality of biomolecules.

For example, in step S501, the processing unit 101 can cause the output unit 104 (particularly, a display device) to display an input reception window for receiving the input to prompt the user to perform the input.

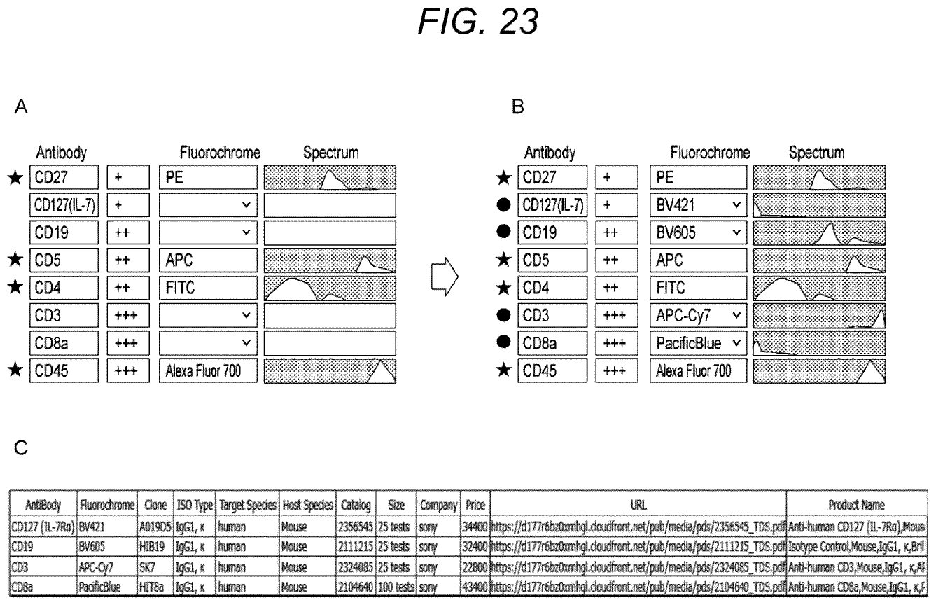

An example of the input reception window is illustrated in A of FIG. 23. The window illustrated in A of FIG. 23 includes a column of a list box for specifying a fluorophore to be assigned to the biomolecule in addition to a column of a list box for receiving the input of the biomolecule and the expression level. For each of the biomolecules “CD27”, “CD5”, “CD4”, and “CD45” described in the list box, a fluorophore is assigned using the fluorophore use history information and/or the in-document fluorophore use result information described in (3-2-3) above. As a result of the assignment, “PE”, “APC”, “FITC”, and “Alexa Fluor 700” were assigned to the biomolecules “CD27”, “CD5”, “CD4”, and “CD 45”, respectively.

In step S502, the processing unit 101 classifies a plurality of biomolecules selected in step S501 on the basis of the expression level selected for each biomolecule, and generates one or a plurality of expression level categories, in particular, a plurality of expression level categories. The biomolecules to which the fluorophores are assigned in step S501 may also be classified on the basis of the expression level in step S502.

The number of expression level categories may be, for example, a value corresponding to the number of expression level levels and may be preferably two to 20, preferably two to 15, even more preferably two to 10, for example, three to 10.

In step S503, the processing unit 101 acquires a list of fluorophores capable of labeling the biomolecules input in step S501. Furthermore, the processing unit 101 also acquires information regarding the fluorophore assigned in step S501. The list of these fluorophores may be acquired, for example, from a database existing outside the information processing device 100, or may be acquired from a database stored inside the information processing device 100 (for example, the storage unit 102).

In step S504, the processing unit 101 classifies the fluorophores included in the list regarding the fluorophores capable of labeling the biomolecules, in the list of the fluorophores acquired in step S503, on the basis of the brightness of each fluorophore, and generates one or a plurality of brightness categories, in particular, a plurality of brightness categories.

In the list of the phosphors acquired in step S503, the fluorophores assigned in step S501 are also classified on the basis of the brightness of each fluorophore and belong to one of the brightness categories.

In step S505, the processing unit 101 associates the expression level categories generated in step S502 with the brightness categories generated in step S504. Preferably, the processing unit 101 associates one expression level category with one brightness category. Furthermore, the processing unit 101 can perform association such that the expression level category and the brightness category correspond on a one-to-one basis. That is, the association can be performed such that two or more expression level categories are not associated with one brightness category.

In step S506, the processing unit 101 specifies an optimal fluorophore combination by using correlation information between fluorophores. The optimal fluorophore combination may be, for example, a fluorophore combination that is optimal from the viewpoint of the correlation between the fluorophore spectra, may be more particularly a fluorophore combination that is optimal from the viewpoint of the correlation coefficient between the fluorophore spectra, and may be even more particularly a fluorophore combination that is optimal from the viewpoint of the square of the correlation coefficient between the fluorophore spectra.

The correlation information between the fluorophores may be preferably correlation information between fluorophore spectra. That is, in the preferred embodiment of the present technology, the processing unit 101 specifies an optimal fluorophore combination by using correlation information between fluorophore spectra.

An example of how to specify the optimal fluorophore combination will be described below.

The processing unit 101 selects the same number of fluorophores as the “number of biomolecules belonging to an expression level category associated with a certain brightness category” from the certain brightness category. However, in a case where the fluorophore assigned in step S501 is included in the certain brightness category, the processing unit 101 selects the same number of fluorophores as (“Number of biomolecules belonging to expression level category associated with certain brightness category”-“Fluorophore input in step S501”) from the certain brightness category.

The selection of fluorophores described above is performed for all brightness categories. Thus, the sum of the “number of selected fluorophores” and “the number of fluorophores input in step S501” is the same as “the number of a plurality of biomolecules used for analysis of a sample”, and in this manner, one fluorophore combination candidate is obtained.

Next, the processing unit 101 calculates the square of the correlation coefficient between the fluorescence spectra for a combination of any two fluorophores included in the fluorophore combination candidate. The processing unit 101 calculates the square of the correlation coefficient for all combinations. For example, through the calculation processing, the processing unit 101 obtains a matrix of correlation coefficient square values as illustrated in FIG. 12. Then, the processing unit 101 specifies the maximum correlation coefficient square value from the matrix of correlation coefficient square values.

Here, in a case where the “number of fluorophores belonging to a certain brightness category” is larger than the “number of biomolecules belonging to an expression level category associated with the certain brightness category”, there is a plurality of combinations of fluorophores selected from the certain brightness category. For example, there are six kinds of fluorophore combinations (=₄C₂) in a case where two fluorophores are selected from four fluorophores. Therefore, for example, in a case where there are three brightness categories, four fluorophores belong to any of the three brightness categories, and two fluorophores are selected from each brightness category, there are 216 fluorophore combination candidates of 6×6×6. However, in a case where the fluorophores input in step S501 are included in the certain brightness category, the number of fluorophore combination candidates decreases.

In the present disclosure, the processing unit 101 specifies the maximum correlation coefficient square value as described above for all possible fluorophore combination candidates. Then, the processing unit 101 specifies a fluorophore combination candidate having the smallest specified maximum correlation coefficient square value. The processing unit 101 specifies the fluorophore combination candidate specified in this manner as an optimal fluorophore combination.

In step S507, the processing unit 101 assigns the fluorophores constituting the optimal fluorophore combination specified in step S506 to the plurality of biomolecules. More specifically, the processing unit 101 assigns each of the fluorophores constituting the optimal fluorophore combination to the biomolecule belonging to the expression level category associated with the brightness category to which the fluorophore belongs.

In a case where two or more fluorophores are included in one brightness category, two or more biomolecules may be included in the associated expression level category. In this case, a fluorophore having a brighter brightness may be assigned to a biomolecule having a lower expression level (or expected to have a lower expression level).

The processing unit 101 generates a combination of a fluorophore and a biomolecule for each biomolecule by the assignment processing described above. In this manner, the processing unit 101 generates a combination list of fluorophores for biomolecules.

In step S508, the processing unit 101 evaluates the separability for the fluorophore group constituting the combination list generated by the assignment processing in step S507. The more detailed processing flow of step S508 is as described with reference to FIG. 18.

However, in step S303 in FIG. 18, the processing unit 101 does not specify the fluorophore assigned in step S501 as a fluorophore having poor separation performance. Thus, it is possible to prevent the fluorophore assigned in step S501 from being changed.

In step S509, the processing unit 101 can cause, for example, the output unit 104 to output the combination list generated in step S508. For example, the combination list can be displayed on the display device.

For example, as illustrated in B of FIG. 23, it is confirmed from the output result that the fluorophore is specified for the circled biomolecule.

Furthermore, in step S509, information regarding a complex of the fluorophore and the biomolecule (for example, information regarding the fluorescently labeled antibody) can be displayed for the biomolecule to which the fluorophore selected by optimization is assigned. An example of how to display such information is illustrated in C of FIG. 23. As illustrated in C of FIG. 23, the information includes a biomolecule name (antigen name captured by an antibody, “Antibody” column), a fluorophore name labeling a biomolecule (“Fluorochrome” column), a clone name (“Clone” column), an isotype (“ISO Type” column), a species from which an antigen captured by the biomolecule (antibody) is derived (“Target Species” column), a species from which the biomolecule (antibody) is derived (“Host Species” column), a catalog number (“Catalog” column), a reagent size (“Size” column), a complex (reagent) manufacturer name (“Company” column), a price (“Price” column), a web page related to the antibody (“URL” column), and a complex product name (“Product Name” column). In the present disclosure, the information regarding the complex may include one or more pieces of the listed information, and preferably includes a biomolecule name and a fluorophore name that labels the biomolecule. In a preferred embodiment, the information may further include a price.

(3-3-5) Still Another Example of Processing (Processing Flow in Case where Inter-Fluorophore Stain Index or Inter-Fluorophore Spillover Spreading Matrix is

### Used as Correlation Information)

In (3-3-1) described above, in step S106, the correlation between the fluorophore spectra has been described as an example of the correlation information between the fluorophores, but in another embodiment of the present disclosure, the correlation information between the fluorophores is not limited thereto. For example, as the correlation information between the fluorophores, an inter-fluorophore stain index or an inter-fluorophore spillover spreading matrix may be used. Other embodiments are as described in (3-3-1) above except that the correlation information to be used is different. For example, in the processing flow described in (3-3-1) above, step S106 is different, but the other steps are the same. Therefore, step S106 will be described below.

### (3-3-5-1) Case of Using Inter-Fluorophore Stain Index

In this case, a standardized inter-fluorophore stain index list may be prepared in advance in order to execute the processing in step S106. In the present description, the standardized inter-fluorophore stain index is also referred to as “standardized inter-fluorophore SI”.

The standardized inter-fluorophore SI list may be a list having inter-fluorophore stain indices for all combinations of two fluorophores in the fluorophore group including at least all the fluorophores included in the list regarding the fluorophores acquired in step S103. For example, the standardized inter-fluorophore stain index list may be a list having inter-fluorophore stain indices for all combinations of two fluorophores in the fluorophore group including all the fluorophores usable in a device where the processing unit 101 can use a combination list of fluorophores for biomolecules.

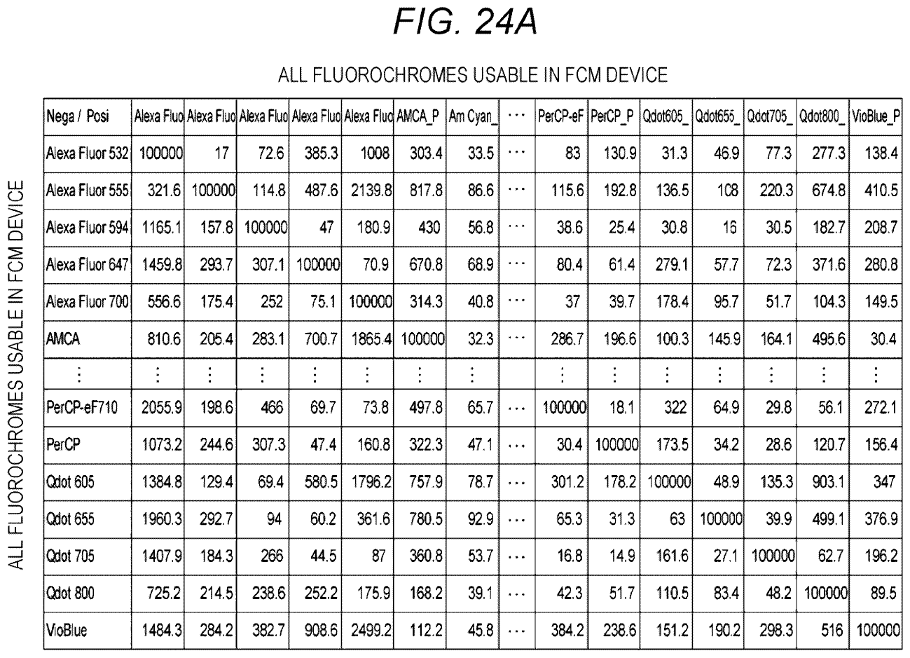

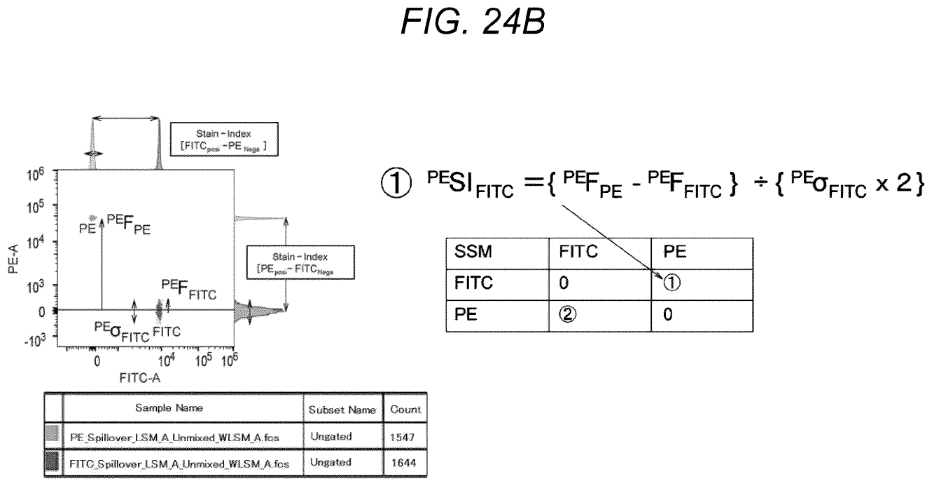

An example of the standardized inter-fluorophore SI list is illustrated in FIG. 24A. As illustrated in FIG. 24A, the top of the list indicates a positive fluorophore and the side of the list indicates a negative fluorophore. For example, all the fluorophores usable in the device are listed on both the top and the side of the list. Note that in FIG. 24A, “. . .” means omission of a part of the list.

A method for calculating the standardized inter-fluorophore SI will be described with reference to FIG. 24B. First, simulation data is generated for all the fluorophores appearing in the standardized inter-fluorophore SI list on the condition that the fluorescence amount of each fluorophore is the same. Then, the standardized inter-fluorophore SI between two different fluorophores is calculated using the simulation data. FIG. 24B illustrates an example of calculation of the standardized inter-fluorophore SI of PE for FITC. The standardized inter-fluorophore SI means separation performance for FITC when PE is positive.

A circled number 1 in FIG. 24B is Equation for calculating the standardized inter-fluorophore SI (^{PE}SI_{FITC}) in a case where PE is positive and FITC is negative. As shown in this Equation, ^{PE}SI_{FITC} is represented by {PEFPE-^{PE}F_{FITC}}÷{^{PE}O_{FITC}×2}. The terms in this Equation are as shown on the left of FIG. 24B and mean the followings. ^{PE}F_{PE} is the average fluorescence intensity of PE-positive and FITC-negative particles at the fluorescence wavelength of PE.

^{PE}F_{FITC} is the average fluorescence intensity of PE-negative and FITC-positive particles at the fluorescence wavelength of PE.

^{PE}O_{FITC} is the standard deviation of the average fluorescence intensity of PE-negative and FITC-positive particles at the fluorescence wavelength of PE.

The above-described calculation is performed for all the combinations of two fluorophores, and the standardized inter-fluorophore SI list as illustrated in FIG. 24A is generated.

In step S106, the processing unit 101 specifies an optimal fluorophore combination by using the standardized inter-fluorophore SI list prepared as described above as the correlation information between the fluorophores. An example of how to specify the optimal fluorophore combination will be described below.

The processing unit 101 selects the same number of fluorophores as the “number of biomolecules belonging to an expression level category associated with a certain brightness category” from the certain brightness category. The selection of fluorophores is performed for all brightness categories. Thus, the same number of fluorophores as the “number of a plurality of biomolecules used for analysis of a sample” are selected, and in this manner, one fluorophore combination candidate is obtained.

Next, for a combination of any two fluorophores included in the fluorophore combination candidate, the processing unit 101 refers to the standardized inter-fluorophore SI list and specifies the standardized inter-fluorophore SI corresponding to the combination of the two fluorophores. Here, for each combination, the standardized inter-fluorophore SI in a case where one is positive and the other is negative, and the standardized inter-fluorophore SI in a case where one is negative and the other is positive are specified. The processing unit 101 specifies such two standardized inter-fluorophore SIs for all the combinations of two fluorophores included in the fluorophore combination candidate. Then, the processing unit 101 specifies the minimum value among all the standardized inter-fluorophore SIs specified for the fluorophore combination candidate.

Note that the larger the standardized inter-fluorophore SI is, the better the separation performance is. Therefore, it is considered that the fluorophore combination candidate obtaining the minimum value has better separation performance as the minimum value specified as described above is larger.

Here, as described in (3-3-1) above, in a case where the “number of fluorophores belonging to a certain brightness category” is larger than the “number of biomolecules belonging to an expression level category associated with the certain brightness category”, there is a plurality of combinations of fluorophores selected from the certain brightness category. In the present disclosure, the processing unit 101 specifies the minimum value of the standardized inter-fluorophore SI as described above for all possible fluorophore combination candidates. Then, the processing unit 101 specifies a fluorophore combination candidate having the largest specified minimum value. The processing unit 101 specifies the fluorophore combination candidate specified in this manner as an optimal fluorophore combination.

### (3-3-5-2) Case of Using Inter-Fluorophore Spillover Spreading Matrix

In this case, an inter-fluorophore spillover spreading matrix may be prepared in advance in order to execute the processing in step S106. In the present description, the inter-fluorophore spillover spreading matrix is also referred to as “inter-fluorophore SSM”. Furthermore, the inter-fluorophore spillover spreading is also referred to as “inter-fluorophore SS”.

The inter-fluorophore SSM may be an inter-fluorophore SSM for all combinations of two fluorophores in the fluorophore group including at least all the fluorophores included in the list regarding the fluorophores acquired in step S103. For example, the inter-fluorophore SSM may be an inter-fluorophore SSM for all combinations of two fluorophores in the fluorophore group including all the fluorophores usable in a device where the processing unit 101 can use a combination list of fluorophores for biomolecules.

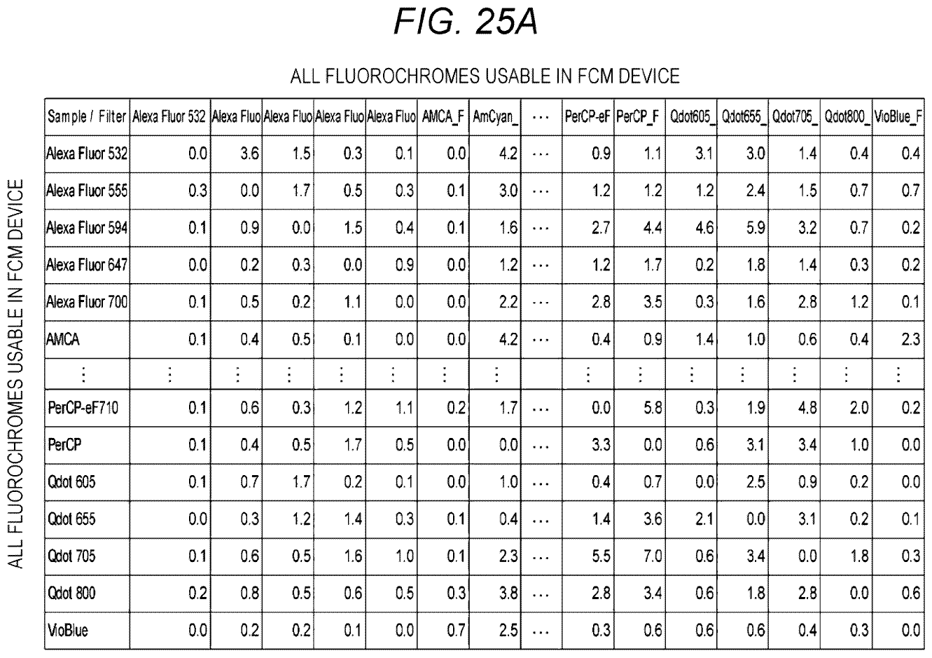

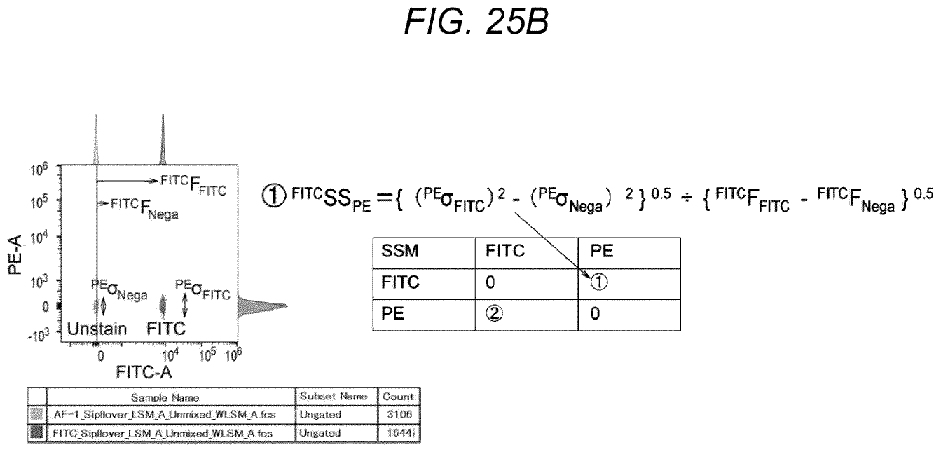

An example of the inter-fluorophore SSM is illustrated in FIG. 25A. As illustrated in FIG. 25A, the top (Filter) of the matrix indicates a detector in a wavelength band corresponding to each fluorophore, and the side (Sample) of the matrix indicates particles labeled with the fluorophores. Each inter-fluorophore SS is an index related to the degree of leakage of light generated from the fluorophore shown in Sample to the detector corresponding to the fluorophore shown in Filter. Note that in FIG. 24A, “ . . . ” means omission of a part of the list.

A method for calculating the inter-fluorophore SS will be described with reference to FIG. 25B. First, simulation data is generated for all the fluorophores appearing in the standardized inter-fluorophore SI list on the condition that the fluorescence amount of each fluorophore is the same. Then, the inter-fluorophore SS between two different fluorophores is calculated using the simulation data. Note that data obtained by actually measuring the fluorescence-labeled particles may be used instead of the simulation data. FIG. 25B illustrates an example of calculation of the inter-fluorophore SS of FITC for PE. The inter-fluorophore SS means the degree of leakage of fluorescence of light generated from FITC to a detector corresponding to PE.

A circled number 1 in FIG. 25B is Equation for calculating the inter-fluorophore SS (^{FITC}SS_{PE}) of FITC for PE. As shown in this Equation, ^{FITC}SS_{PE} is represented by {(^{PE}σ_{FITC})²−(^{PE}σ_{Nega})²}^{0.5}÷{^{FITC}F_{FITC}−^{FITC}F_{Nega}}^{0.5}. The terms in this Equation are as shown on the left of FIG. 24B and mean the followings.

^{PE}σ_{FITC} is the standard deviation of the average fluorescence intensity of PE-negative and FITC-positive particles at the fluorescence wavelength of PE.

^{PE}σ_{Nega} is the standard deviation of the average fluorescence intensity of PE-negative and FITC-negative particles at the fluorescence wavelength of PE.

^{FITC}F_{FITC} is the average fluorescence intensity of PE-negative and FITC-positive particles at the fluorescence wavelength of FITC.

^{FITC}F_{Nega} is the average fluorescence intensity of PE-negative and FITC-negative particles at the fluorescence wavelength of FITC.

The above-described calculation is performed for all the combinations of two fluorophores, and the inter-fluorophore SSM as illustrated in FIG. 25A is generated.

In step S106, the processing unit 101 specifies an optimal fluorophore combination by using the inter-fluorophore SSM prepared as described above as the correlation information between the fluorophores. An example of how to specify the optimal fluorophore combination will be described below.

The processing unit 101 selects the same number of fluorophores as the “number of biomolecules belonging to an expression level category associated with a certain brightness category” from the certain brightness category. The selection of fluorophores is performed for all brightness categories. Thus, the same number of fluorophores as the “number of a plurality of biomolecules used for analysis of a sample” are selected, and in this manner, one fluorophore combination candidate is obtained.

Next, for a combination of any two fluorophores included in the fluorophore combination candidate, the processing unit 101 refers to the inter-fluorophore SSM and specifies the inter-fluorophore SS corresponding to the combination of the two fluorophores. Here, for each combination, the inter-fluorophore SS in a case where one is positive and the other is negative, and the inter-fluorophore SS in a case where one is negative and the other is positive are specified. The processing unit 101 specifies such two inter-fluorophore SSs for all combinations of two fluorophores included in the fluorophore combination candidate. Then, the processing unit 101 specifies the maximum value among all the inter-fluorophore SSs specified for the fluorophore combination candidate.

Note that the smaller the inter-fluorophore SS is, the better the separation performance is. Therefore, it is considered that the fluorophore combination candidate obtaining the maximum value has better separation performance as the maximum value specified as described above is smaller.

Here, as described in (3-3-1) above, in a case where the “number of fluorophores belonging to a certain brightness category” is larger than the “number of biomolecules belonging to an expression level category associated with the certain brightness category”, there is a plurality of combinations of fluorophores selected from the certain brightness category. In the present disclosure, the processing unit 101 specifies the maximum value of the inter-fluorophore SS as described above for all possible fluorophore combination candidates. Then, the processing unit 101 specifies a fluorophore combination candidate having the smallest specified maximum value. The processing unit 101 specifies the fluorophore combination candidate specified in this manner as an optimal fluorophore combination.

### (3-4) Configuration Example of Biological Sample Analyzer

A configuration example of a biological sample analyzer that executes analysis using a fluorophore-labeled biomolecule capturing substance (for example, a fluorochrome-labeled antibody) obtained according to a combination list generated using the information processing device according to the present disclosure will be described below.

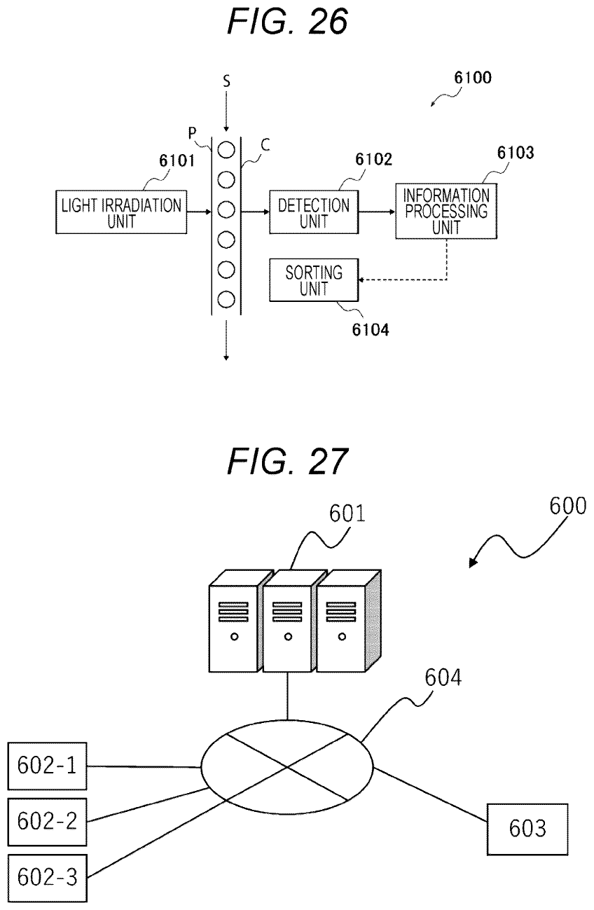

The configuration example of the biological sample analyzer is illustrated in FIG. 26. A biological sample analyzer 6100 illustrated in FIG. 26 includes: a light irradiation unit 6101 that irradiates a biological sample S flowing in a flow channel C with light; a detection unit 6102 that detects light generated by irradiating the biological sample S with light; and an information processing unit 6103 that processes information regarding the light detected by the detection unit. The biological sample analyzer 6100 is a flow cytometer or an imaging cytometer, for example. The biological sample analyzer 6100 may include a sorting unit 6104 that sorts out specific biological particles P in a biological sample. The biological sample analyzer 6100 including the sorting unit is a cell sorter, for example.

### (Biological Sample)

The biological sample S may be a liquid sample containing biological particles. The biological particles are cells or non-cellular biological particles, for example. The cells may be living cells, and more specific examples thereof include blood cells such as erythrocytes and leukocytes, and germ cells such as sperms and fertilized eggs. Also, the cells may be those directly collected from a sample such as whole blood, or may be cultured cells obtained after culturing. The non-cellular biological particles are extracellular vesicles, or particularly, exosomes and microvesicles, for example. The biological particles may be labeled with one or more labeling substances (such as a dye (particularly, a fluorochrome) and a fluorochrome-labeled antibody). Note that particles other than biological particles may be analyzed by the biological sample analyzer of the present disclosure, and beads or the like may be analyzed for calibration or the like.

### (Flow Channel)

The flow channel C is designed so that a flow of the biological sample S is formed. In particular, the flow channel C may be designed so that a flow in which the biological particles contained in the biological sample are aligned substantially in one row is formed. The flow channel structure including the flow channel C may be designed so that a laminar flow is formed. In particular, the flow channel structure is designed so that a laminar flow in which the flow of the biological sample (a sample flow) is surrounded by the flow of a sheath liquid is formed. The design of the flow channel structure may be appropriately selected by a person skilled in the art, or a known one may be adopted. The flow channel C may be formed in a flow channel structure such as a microchip (a chip having a flow channel on the order of micrometers) or a flow cell. The width of the flow channel C is 1 mm or smaller, or particularly, may be not smaller than 10 μm and not greater than 1 mm. The flow channel C and the flow channel structure including the flow channel C may be made of a material such as plastic or glass.

The biological sample analyzer of the present disclosure is designed so that the biological sample flowing in the flow channel C, or particularly, the biological particles in the biological sample are irradiated with light from the light irradiation unit 6101. The biological sample analyzer of the present disclosure may be designed so that the irradiation point of light on the biological sample is located in the flow channel structure in which the flow channel C is formed, or may be designed so that the irradiation point is located outside the flow channel structure. An example of the former case may be a configuration in which the light is emitted onto the flow channel C in a microchip or a flow cell. In the latter case, the biological particles after exiting the flow channel structure (particularly, the nozzle portion thereof) may be irradiated with the light, and a flow cytometer of a jet-in-air type can be adopted, for example.

### (Light Irradiation Unit)

The light irradiation unit 6101 includes a light source unit that emits light, and a light guide optical system that guides the light to the irradiation point. The light source unit includes one or more light sources. The type of the light source(s) is a laser light source or an LED, for example. The wavelength of light to be emitted from each light source may be any wavelength of ultraviolet light, visible light, and infrared light. The light guide optical system includes optical components such as beam splitters, mirrors, or optical fibers, for example. The light guide optical system may also include a lens group for condensing light, and includes an objective lens, for example. There may be one or more irradiation points at which the biological sample and light intersect. The light irradiation unit 6101 may be designed to collect light emitted onto one irradiation point from one light source or different light sources.

### (Detection Unit)

The detection unit 6102 includes at least one photodetector that detects light generated by emitting light onto biological particles. The light to be detected may be fluorescence or scattered light (such as one or more of the following: forward scattered light, backscattered light, and side scattered light), for example. Each photodetector includes one or more light receiving elements, and has a light receiving element array, for example. Each photodetector may include one or more photomultiplier tubes (PMTs) and/or photodiodes such as APDs and MPPCs, as the light receiving elements. The photodetector includes a PMT array in which a plurality of PMTs is arranged in a one-dimensional direction, for example. The detection unit 6102 may also include an image sensor such as a CCD or a CMOS. With the image sensor, the detection unit 6102 can acquire an image (such as a bright-field image, a dark-field image, or a fluorescent image, for example) of biological particles.

The detection unit 6102 includes a detection optical system that causes light of a predetermined detection wavelength to reach the corresponding photodetector. The detection optical system includes a spectroscopic unit such as a prism or a diffraction grating, or a wavelength separation unit such as a dichroic mirror or an optical filter. The detection optical system is designed to disperse the light generated by light irradiation to biological particles, for example, and detect the dispersed light with a larger number of photodetectors than the number of fluorochromes with which the biological particles are labeled. A flow cytometer including such a detection optical system is called a spectral flow cytometer. Further, the detection optical system is designed to separate the light corresponding to the fluorescence wavelength band of a specific fluorochrome from the light generated by the light irradiation to the biological particles, for example, and cause the corresponding photodetector to detect the separated light.

The detection unit 6102 may also include a signal processing unit that converts an electrical signal obtained by a photodetector into a digital signal. The signal processing unit may include an A/D converter as a device that performs the conversion. The digital signal obtained by the conversion performed by the signal processing unit can be transmitted to the information processing unit 6103. The digital signal can be handled as data related to light (hereinafter, also referred to as “light data”) by the information processing unit 6103. The light data may be light data including fluorescence data, for example. More specifically, the light data may be data of light intensity, and the light intensity may be light intensity data of light including fluorescence (the light intensity data may include feature quantities such as area, height, and width).

### (Information Processing Unit)

The information processing unit 6103 includes a processing unit that performs processing of various kinds of data (light data, for example), and a storage unit that stores various kinds of data, for example. In a case where the processing unit acquires the light data corresponding to a fluorochrome from the detection unit 6102, the processing unit can perform fluorescence leakage correction (a compensation process) on the light intensity data. In the case of a spectral flow cytometer, the processing unit also performs a fluorescence separation processing on the light data, and acquires the light intensity data corresponding to the fluorochrome. The fluorescence separation process may be performed by an unmixing method disclosed in JP 2011-232259 A, for example. In a case where the detection unit 6102 includes an image sensor, the processing unit may acquire morphological information regarding the biological particles, on the basis of an image acquired by the image sensor. The storage unit may be designed to be capable of storing the acquired light data. The storage unit may be designed to be capable of further storing spectral reference data to be used in the unmixing process.

In a case where the biological sample analyzer 6100 includes the sorting unit 6104 described later, the information processing unit 6103 can determine whether to sort the biological particles, on the basis of the light data and/or the morphological information. The information processing unit 6103 then controls the sorting unit 6104 on the basis of the result of the determination, and the biological particles can be sorted by the sorting unit 6104.

The information processing unit 6103 may be designed to be capable of outputting various kinds of data (such as light data and images, for example). For example, the information processing unit 6103 can output various kinds of data (such as a two-dimensional plot or a spectrum plot, for example) generated on the basis of the light data. The information processing unit 6103 may also be designed to be capable of accepting inputs of various kinds of data, and accepts a gating processing on a plot by a user, for example. The information processing unit 6103 may include an output unit (such as a display, for example) or an input unit (such as a keyboard, for example) for performing the output or the input.

The information processing unit 6103 may be designed as a general-purpose computer, and may be designed as an information processing device that includes a CPU, a RAM, and a ROM, for example. The information processing unit 6103 may be included in the housing in which the light irradiation unit 6101 and the detection unit 6102 are included, or may be located outside the housing. Further, the various processes or functions to be executed by the information processing unit 6103 may be realized by a server computer or a cloud connected via a network.

### (Sorting Unit)

The sorting unit 6104 performs sorting of biological particles, in accordance with the result of determination performed by the information processing unit 6103. The sorting method may be a method by which droplets containing biological particles are generated by vibration, electric charges are applied to the droplets to be sorted, and the traveling direction of the droplets is controlled by an electrode. The sorting method may be a method for sorting by controlling the traveling direction of biological particles in the flow channel structure. The flow channel structure has a control mechanism based on pressure (injection or suction) or electric charge, for example. An example of the flow channel structure may be a chip (the chip disclosed in JP 2020-76736 A, for example) that has a flow channel structure in which the flow channel C branches into a recovery flow channel and a waste liquid flow channel on the downstream side, and specific biological particles are collected in the recovery flow channel.

Furthermore, the biological sample analyzer described above may be configured as the information processing device according to the present disclosure. For example, the information processing unit 6103 may function as the processing unit 101 according to the present disclosure, and may be configured to execute the processing described in (3-2) or (3-3) above.

### 2. Second Embodiment (Information Processing System)

The present disclosure also provides an information processing system including the processing unit described in “1. First Embodiment (Information Processing Device)”. In addition to the processing unit, the information processing system may include the storage unit, the input unit, the output unit, and the communication unit, which are described above in “1. First Embodiment (Information Processing Device)”. These components may be provided in one device or may be provided in a plurality of devices in a distributed manner.

The information processing system according to the present disclosure also enables an appropriate panel design as described above in “1. First Embodiment (Information Processing Device)”.

A configuration example of the information processing system according to the present disclosure will be described with reference to FIG. 27. An information processing system 600 illustrated in FIG. 27 includes an information processing device 601, biological sample analyzers 602-1 to 602-3, and a terminal 603. These devices included in the information processing system 600 may be connected via a network 604.

The information processing device 601 may be configured as described in “1. First Embodiment (Information Processing Device)”. The information processing device 601 may be configured by, for example, a server computer. The information processing device 601 may be configured to automatically collect a combination list of biomolecules and fluorophores, which is used in the biological sample analysis by the biological sample analyzers 602-1 to 602-3. The combination list can be used as the fluorophore use history information.

The biological sample analyzers 602-1 to 602-3 may be configured as described in (3-4) above, and may be, for example, a flow cytometer. The biological sample analyzers 602-1 to 602-3 may be closed cell sorters. The number of biological sample analyzers that can be connected to the information processing system 600 is not limited to three as illustrated in FIG. 27, and may be one or more, in particular, plural. For example, the biological sample analyzers 602-1 to 602-3 may be configured to transmit the combination list of the biomolecules and the fluorophores, which is used in the biological sample analysis to the information processing device 601 when the device performs the biological sample analysis, and the transmission may be automatically performed.

The terminal 603 is a device operated by a user who executes information processing according to the present disclosure, and the terminal may be configured as an information processing device. The information processing device may be configured as described in (3-1) above, and may be a desktop terminal, a laptop terminal, or a tablet terminal.

In the embodiment, the information processing device 601 can execute the processing described in (3-2) or (3-3) above, and the terminal 603 can display various windows and the like generated in accordance with the processing.

In another embodiment, the terminal 603 may execute the processing described in (3-2) or (3-3) above, and in the processing, the fluorophore use history information and/or the in-document fluorophore use result information stored in the information processing device 601 may be acquired or referred to.

### 3. Third Embodiment (Information Processing Method)

The present disclosure relates to an information processing method. The information processing method may include presentation processing of presenting a fluorophore candidate that can be assigned to a biomolecule on the basis of fluorophore use history information, in-document fluorophore use result information, or both of them. The presentation processing may be executed as described in (3-2-3-1) and/or (3-2-3-2) above. Execution of the presentation processing enables efficient generation of an appropriate panel.

Furthermore, the information processing method according to the present disclosure may include acquisition processing of acquiring fluorophore use history information and/or in-document fluorophore use result information.

For example, the presentation processing may be executed as a part of processing of generating a combination list of fluorophores for biomolecules. The generation processing may be executed as described in (3-2) above. Furthermore, the information processing method may include processing by an algorithm in addition to the presentation processing. The processing by the algorithm may be executed as described in (3-3) above. The information processing method according to the present disclosure enables efficient generation of an appropriate panel.

### 4. Fourth Embodiment (Program)

The present disclosure also provides a program for causing the information processing device to execute the information processing method described in the above-described 3. The information processing method is as described in the above-described 1, and 3., and the description also applies to the present embodiment. The program according to the present disclosure may be recorded in, for example, the recording medium described above, or may be stored in the information processing device described above or a storage unit included in the information processing device described above.

Note that the present disclosure can also have the following configurations.

(1)

An information processing device including
a processing unit configured to present a fluorophore candidate that is capable of being assigned to a biomolecule on the basis of fluorophore use history information, in-document fluorophore use result information, or both of the fluorophore use history information and the in-document fluorophore use result information.
   (2)

The information processing device according to (1),
in which the fluorophore use history information includes a combination list of biomolecules and fluorophores, the combination list being used in biological sample analysis.
   (3)

The information processing device according to (1) or (2),
in which the in-document fluorophore use result information includes a combination list of biomolecules and fluorophores, the combination list being described in a document.
   (4)

The information processing device according to any one of (1) to (3),
in which the processing unit is configured to cause an output unit to display a selection window that prompts selection of a biomolecule to be analyzed in biological sample analysis, and
the selection window is configured to select an analysis target biomolecule from among the biomolecules included in the fluorophore use history information, the in-document fluorophore use result information, or both of the fluorophore use history information and the in-document fluorophore use result information.
   (5)

The information processing device according to (4),
in which the processing unit causes the output unit to display a fluorophore candidate that is capable of being assigned to the biomolecule selected in the selection window on the basis of the fluorophore use history information, the in-document fluorophore use result information, or both of the fluorophore use history information and the in-document fluorophore use result information.
   (6)

The information processing device according to (5),
in which the processing unit is configured to generate a combination list of fluorophores for biomolecules, and
the combination list includes a combination of the selected biomolecule and the fluorophore selected from among the fluorophore candidates.
   (7)

The information processing device according to (6),
in which the combination list includes a combination of the biomolecule and the fluorophore, which is specified by the processing unit, in addition to the combination of the selected biomolecule and the fluorophore selected from among the fluorophore candidates.
   (8)

The information processing device according to any one of (1) to (7),
in which the processing unit is configured to perform search for a biomolecule to be analyzed in biological sample analysis, and
the processing unit searches the fluorophore use history information, the in-document fluorophore use result information, or both of the fluorophore use history information and the in-document fluorophore use result information on the basis of an input keyword.
   (9)

The information processing device according to any one of (1) to (8),
in which the fluorophore use history information, the in-document fluorophore use result information, or both of the fluorophore use history information and the in-document fluorophore use result information are associated with user information.
   (10)

The information processing device according to any one of (1) to (9),
in which the information processing device is configured to automatically collect a combination list of biomolecules and fluorophores, which is used in analysis processing of a biological sample analyzer, as the fluorophore use history information.
   (11)

The information processing device according to any one of (1) to (10),
in which the information processing device is configured to display a window that receives an input of the fluorophore use history information, a window that receives an input of the in-document fluorophore use result information, or a window that receives an input of both of the fluorophore use history information and the in-document fluorophore use result information.
   (12)

The information processing device according to any one of (1) to (11),
in which the information processing device includes a storage unit configured to store the fluorophore use history information, the in-document fluorophore use result information or both of the fluorophore use history information and the in-document fluorophore use result information.
   (13)

The information processing device according to any one of (1) to (12),
in which the processing unit is configured to generate a combination list of fluorophores for biomolecules, and
the processing unit is configured to display an input box regarding whether to include a tandem dye in the combination list.
   (14)

The information processing device according to any one of (1) to (13),
in which the information processing device includes a storage unit configured to store stock information of a fluorophore, price information, or both of the stock information and the price information.
   (15)

An information processing system including
a processing unit configured to present a fluorophore candidate that is capable of being assigned to a biomolecule on the basis of fluorophore use history information, in-document fluorophore use result information, or both of the fluorophore use history information and the in-document fluorophore use result information.

### REFERENCE SIGNS LIST

100 Information processing device
101 Processing unit
102 Storage unit
103 Input unit
104 Output unit
105 Communication unit

1. An information processing device comprising
a processing unit configured to present a fluorophore candidate that is capable of being assigned to a biomolecule on a basis of fluorophore use history information, in-document fluorophore use result information, or both of the fluorophore use history information and the in-document fluorophore use result information.

2. The information processing device according to claim 1, wherein the fluorophore use history information includes a combination list of biomolecules and fluorophores, the combination list being used in biological sample analysis.

3. The information processing device according to claim 1, wherein the in-document fluorophore use result information includes a combination list of biomolecules and fluorophores, the combination list being described in a document.

4. The information processing device according to claim 1, wherein
the processing unit is configured to cause an output unit to display a selection window that prompts selection of a biomolecule to be analyzed in biological sample analysis, and
the selection window is configured to select an analysis target biomolecule from among the biomolecules included in the fluorophore use history information, the in-document fluorophore use result information, or both of the fluorophore use history information and the in-document fluorophore use result information.

5. The information processing device according to claim 4, wherein the processing unit causes the output unit to display a fluorophore candidate that is capable of being assigned to the biomolecule selected in the selection window on a basis of the fluorophore use history information, the in-document fluorophore use result information, or both of the fluorophore use history information and the in-document fluorophore use result information.

6. The information processing device according to claim 5, wherein
the processing unit is configured to generate a combination list of fluorophores for biomolecules, and
the combination list includes a combination of the selected biomolecule and the fluorophore selected from among the fluorophore candidates.

7. The information processing device according to claim 6, wherein
the combination list includes a combination of the biomolecule and the fluorophore, which is specified by the processing unit, in addition to the combination of the selected biomolecule and the fluorophore selected from among the fluorophore candidates.

8. The information processing device according to claim 1, wherein
the processing unit is configured to perform search for a biomolecule to be analyzed in biological sample analysis, and
the processing unit performs search for the fluorophore use history information, the in-document fluorophore use result information, or both of the fluorophore use history information and the in-document fluorophore use result information on a basis of an input keyword.

9. The information processing device according to claim 1, wherein the fluorophore use history information, the in-document fluorophore use result information, or both of the fluorophore use history information and the in-document fluorophore use result information are associated with user information.

10. The information processing device according to claim 1, wherein the information processing device is configured to automatically collect a combination list of biomolecules and fluorophores, which is used in analysis processing of a biological sample analyzer, as the fluorophore use history information.

11. The information processing device according to claim 1, wherein the information processing device is configured to display a window that receives an input of the fluorophore use history information, a window that receives an input of the in-document fluorophore use result information, or a window that receives an input of both of the fluorophore use history information and the in-document fluorophore use result information.

12. The information processing device according to claim 1, wherein the information processing device includes a storage unit configured to store the fluorophore use history information, the in-document fluorophore use result information or both of the fluorophore use history information and the in-document fluorophore use result information.

13. The information processing device according to claim 1, wherein
the processing unit is configured to generate a combination list of fluorophores for biomolecules, and
the processing unit is configured to display an input box regarding whether to include a tandem dye in the combination list.

14. The information processing device according to claim 1, wherein the information processing device includes a storage unit configured to store stock information of a fluorophore, price information, or both of the stock information and the price information.

15. An information processing system comprising
a processing unit configured to present a fluorophore candidate that is capable of being assigned to a biomolecule on a basis of fluorophore use history information, in-document fluorophore use result information, or both of the fluorophore use history information and the in-document fluorophore use result information.
